SEQUENCE LISTING (36 sequences)

SEQ ID NO: 1 - DNA, 20 bp, Artificial sequence
  Synthetic sequence Primer
caggtgtttgagggtagctc

SEQ ID NO: 2 - DNA, 20 bp, Artificial sequence
  Synthetic sequence Primer
cggttcatcatggtacagtc

SEQ ID NO: 3 - DNA, 23 bp, Artificial sequence
  Synthetic sequence Primer
tctttccaccaggcccccggctc

SEQ ID NO: 4 - DNA, 23 bp, Artificial sequence
  Synthetic sequence Primer
tgcgggcggacatggggagatcc

SEQ ID NO: 5 - DNA, 20 bp, Artificial sequence
  Synthetic sequence Primer
gtgactgcctaccagaatga

SEQ ID NO: 6 - DNA, 20 bp, Artificial sequence
  Synthetic sequence Primer
attgtccgcataggttggag

SEQ ID NO: 7 - DNA, 20 bp, Artificial sequence
  Synthetic sequence Primer
cacgagggacagtcttctgg

SEQ ID NO: 8 - DNA, 20 bp, Artificial sequence
  Synthetic sequence Primer
cgtcggtaaagaaaggcaca

SEQ ID NO: 9 - DNA, 20 bp, Artificial sequence
  Synthetic sequence Primer
ttggggcgagctcattactt

SEQ ID NO: 10 - DNA, 20 bp, Artificial sequence
  Synthetic sequence Primer
ttgccacactctgcacacac

SEQ ID NO: 11 - DNA, 20 bp, Artificial sequence
  Synthetic sequence Primer
gggtaagacccgagttcctc

SEQ ID NO: 12 - DNA, 20 bp, Artificial sequence
  Synthetic sequence Primer
atcaccactttgccaccttc

SEQ ID NO: 13 - DNA, 20 bp, Artificial sequence
  Synthetic sequence Primer
cccactaacatcaaatgggg

SEQ ID NO: 14 - DNA, 20 bp, Artificial sequence
  Synthetic sequence Primer
ccttccacaatgccaaagtt

SEQ ID NO: 15 - DNA, 22 bp, Artificial sequence
  Synthetic sequence Primer
tggtacgggaaatcacaagttt

SEQ ID NO: 16 - DNA, 22 bp, Artificial sequence
  Synthetic sequence Primer
aagcttagccaggttcgagaat

SEQ ID NO: 17 - DNA, 22 bp, Artificial sequence
  Synthetic sequence Primer
tggtacgggaaatcacaagttt

SEQ ID NO: 18 - DNA, 21 bp, Artificial sequence
  Synthetic sequence Primer
agttgtgcatcttggggttct

SEQ ID NO: 19 - DNA, 22 bp, Artificial sequence
  Synthetic sequence Primer
tggtacgggaaatcacaagttt

SEQ ID NO: 20 - DNA, 22 bp, Artificial sequence
  Synthetic sequence Primer
cgcttcatgtgagagagttcct

SEQ ID NO: 21 - DNA, 22 bp, Artificial sequence
  Synthetic sequence Primer
taaggatcccagtgtggtggta

SEQ ID NO: 22 - DNA, 22 bp, Artificial sequence
  Synthetic sequence Primer
tctgctgttgctggtgatagaa

SEQ ID NO: 23 - DNA, 23 bp, Artificial sequence
  Synthetic sequence Primer
cttcggactccggaggaccttct

SEQ ID NO: 24 - DNA, 23 bp, Artificial sequence
  Synthetic sequence Primer
gccaccgattcctggtgggttag

SEQ ID NO: 25 - DNA, 22 bp, Artificial sequence
  Synthetic sequence Primer
atggctgtcagcgacgctctgc

SEQ ID NO: 26 - DNA, 25 bp, Artificial sequence
  Synthetic sequence Primer
catatccagtcactatggctccacc

SEQ ID NO: 27 - DNA, 24 bp, Artificial sequence
  Synthetic sequence Primer
tccgaggacgtcatcaaggagttc

SEQ ID NO: 28 - DNA, 26 bp, Artificial sequence
  Synthetic sequence Primer
ccgatgaacttcaccttgtagatgaa

SEQ ID NO: 29 - DNA, 30 bp, Artificial sequence
  Synthetic sequence Primer
cagtgacacttaccgcattgacaagcacgc

SEQ ID NO: 30 - DNA, 30 bp, Artificial sequence
  Synthetic sequence Primer
gagagagcaatatttcaagaatgcatgcgt

SEQ ID NO: 31 - DNA, 30 bp, Artificial sequence
  Synthetic sequence Primer
cctcgatatacagaccgataaaacacatgc

SEQ ID NO: 32 - DNA, 30 bp, Artificial sequence
  Synthetic sequence Primer
acgcatgattatctttaacgtacgtcacaa

SEQ ID NO: 33 - DNA, 25 bp, Artificial sequence
  Synthetic sequence Primer
atggctgtcagcgacgctctgctcc

SEQ ID NO: 34 - DNA, 27 bp, Artificial sequence
  Synthetic sequence Primer
caccgattcctggtgggttagcgagtt

SEQ ID NO: 35 - DNA, 26 bp, Artificial sequence
  Synthetic sequence Primer
tttaaccctagaaagataatcatatt

SEQ ID NO: 36 - DNA, 23 bp, Artificial sequence
  Synthetic sequence Primer
ttaaccctagaaagatagtctgc

(19) United States
(12) Patent Application Publication
(10) Pub. No.: US 2010/0330677 A1
(43) Pub. Date: Dec. 30, 2010

(54) Improved Reprogramming of Mammalian Cells, and Cells Obtained

(75) Inventor: Austin Gerard Smith, Cambridge Cambridgeshire (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Cambridge Enterprise Limited, Cambridge (GB)

(21) Appl. No.: 12/866,993

(22) PCT Filed: Feb. 11, 2009
(86) PCT No.: PCT/GB2009/000388

§ 371 Date: Aug. 10, 2010

(30) Foreign Application Priority Data
Feb. 11, 2008 (GB) 0802501.7
Mar. 26, 2008 (GB) 0805508.9
Oct. 21, 2008 (GB) 0819324.5

(51) Int. Cl. C12N 15/85; C12N 15/63; C12N 5/10; C12N 5/02

(52) U.S. Cl. 435/455; 435/440; 435/354; 435/366; 435/404

(57) ABSTRACT

Expression of reprogramming factors such as Sox2, klf4, c-myc, Nanog, LIN28 and Oct4 followed by culture in a MEK inhibitor and a GSK3 inhibitor reprograms tissue cells. The invention provides new uses of these inhibitors, for example in inducing completion of the transcriptional resetting of so-called pre-pluripotent (pre-iPS) stem cells, for example as obtained from mammalian neural stem cells or epiblast stem cells treated with single or combinations of the reprogramming factors, expressed transiently or by integrative vectors. Also provided are systems for reprogramming an epiplast stem cells independently of the use of there inhibitors.

### TECHNICAL FIELD

The present invention relates to reprogramming cells to a pluripotent state, to reprogrammed cells obtained thereby and to pluripotent cells per se.

### BACKGROUND ART

Stem cell-based technologies have been identified as offering huge potential for therapeutic and non-therapeutic applications. Much work is currently focused on identifying the true characterising features of various different types of stem cell, including pluripotent stem cells such as embryonic (ES) cells, in particular from humans.

ES cells can be obtained from human embryos but this raises a number of highly sensitive ethical considerations. In many countries such an approach, in addition, is prohibited by law.

An alternative approach, in vitro reprogramming of somatic cells to yield so-called reprogrammed cells, both mouse and human, has been achieved by a number of groups. Initial work by Yamanaka et al (see e.g. Takahashi and Yamanaka, 2006, Cell 126, pp 663-676) has been followed by several others (see e.g. Yu et al, 20 Nov. 2007, Sciencexpress, 10.1126, pp 1-4 and Meissner et al, Nature Biotechnology, vol. 25, no. 10, October 2007, pp 1177-1181).

WO 2007/069666 (also published as EP 1 970 446) describes the Yamanaka et al work, in which a differentiated human cell is reprogrammed into a pluripotent state, the resultant cell being referred to as an induced pluripotent stem cell (iPS). Reprogramming is achieved by using retroviruses to insert a combination of genes which achieve reprogramming, specifically Oct3/4, Sox2, Klf4 and c-Myc.

While this approach has been used by a number of scientists, demonstration of complete reprogramming of a human tissue cell back to a pluripotent cell has not been conclusive. An aim of the technology is to obtain pluripotent cells which can then be differentiated to form specific, desired differentiated cells. The technique has the disadvantage, however, that the reprogramming factors are continually expressed in the cell. This may adversely affect the prospects for successful differentiation unless there is a further step of genetic intervention to silence the expression. Another difficulty with the approach is that the resultant reprogrammed cell is genetically modified, containing retroviral inserts, even if the gene expression is subsequently silenced. These modifications are clinically unacceptable.

To date, the efficiency of reprogramming has been low, often less than 1%. There is also the doubt mentioned above as to whether reprogramming has been complete; alleged iPS cells have shown aberrant gene expression and epigenetic abnormalities. It is suggested that to improve the efficiency additional genes should be introduced into the cell by retroviral integration.

Further, reprogramming occurs over an extended time period. Meissner's group, for example, reported the following, teaching that extended expression of reprogramming factors is required:—
“It appears that ectopic expression of Oct4, Sox2, c-myc and Klf4 initiates a gradual reprogramming process in multiple infected cells that ultimately leads to pluripotency over a time period of several weeks. Using Oct4 EGFP MEFs to monitor reactivation of the endogenous Oct4 locus, we found that all colonies but one were EGFP negative at the time of picking and became EGFP positive only after several passages. This suggests that reprogramming is a slow process involving the sequential activation of ES-cell markers such as AP, SSEA1 and Nanog, with Oct4 activation representing one of the last epigenetic events in the process. Also, these observations are consistent with our previous finding that the numbers of reprogrammed colonies were lower when drug selection for Oct4 activation was applied early after viral transduction, but was substantially higher when drug selection was initiated later. Finally, the slow reprogramming process induced by factor transduction may explain why drug selection for Fbx15 activation as early as 3 d after infection, as used in the initial iPS isolation protocol, yielded only cells that had undergone incomplete epigenetic reprogramming.”

WO2007113505 relates to a serum-free culture medium comprising a MEK inhibitor, a GSK3 inhibitor and, optionally, an antagonist of an FGF receptor which may be used to maintain pluripotent cells in a self-renewing state.

Li et al. (Cell Stem Cell (2009), doi:10.1016/j.stem.2008.11.014) discuss the generation of rat and human “induced pluripotent stem cells” by combining genetic reprogramming and chemical Inhibitors. The same report is discussed in the same journal by Trounson (“Rats, Cats, and Elephants, but Still No Unicorn: Induced Pluripotent Stem Cells from New Species”, Cell Stem Cell (2009), doi:10.1016/j.stem.2008.12.002).

### DISCLOSURE OF THE INVENTION

The present invention aims to address one or more of the inefficiencies or other problems referred to in the cited art above, and an object of the invention is to provide an alternative method for reprogramming mammalian cells. In preferred embodiments of the invention, it is an object to provide improved reprogramming of mammalian cells, in particular human cells, to a pluripotent state.

In various aspects of the invention described below the inventors have used tissue stem cells (such as neural stem cells—“NS cells”) and shown that they appear to be subject to less-stringent epigenetic restrictions than other cells and therefore more amenable to reprogramming. Brain-derived NS cells were able to acquire undifferentiated morphology rapidly and at high frequency after a single round of transduction with reprogramming factors.

Thus, as described below, the present inventors have shown that adult rodent and human NS cells (Conti, 2005) transfected with reprogramming factors rapidly and efficiently acquire features of iPS cells but that in conventional culture conditions reprogramming is stalled and cells do not attain several key attributes of authentic pluripotency. This intermediate cell state is in some respects more like the epithelialised epiblast stem cells (“epistem cells”; EpiSCs) than true ES cells.

These so-called “intermediate” (I-iPS) or pre-pluripotent (pre-iPS) (the terms are used interchangeably) cells are characterized by the incomplete expression of pluripotency associated genes, non-responsiveness to LIF, and retention of epigenetic silencing of the X chromosome. They resemble the original Fbx15 iPS cells described by Takahashi et al. (2006) with very low levels of both Rex1 and Nanog and little or no ability to contribute to chimaeras. This pre-iPS cell state is surprisingly stable and can be propagated extensively and clonally without spontaneous transition to authentic pluripotency.

However the present inventors have further demonstrated that inhibition of one or more intracellular signalling cascades such as pERK and use of LIF (which stimulates Stat3) can actually induce completion of the transcriptional resetting of these intermediate cells and generate authentic pluripotent cells, phenotypically indistinguishable from ES cells by a number of key criteria.

The present inventors have further demonstrated that appropriate use of factors (as described herein) and inhibitors can likewise be employed to convert EpiSCs to ground state using only a single transgene, which can if desired subsequently be removed. For example (re)-expression of Klf4 in combination with an appropriate environment can achieve this. In particular, reprogramming appears to be dependent on inhibition of extrinsic growth factor stimuli. This substantiates the argument that EpiSCs are developmentally, epigenetically and functionally differentiated from ES cells.

Thus disclosed herein are methods of promoting reprogramming of a cell to a (more authentic) pluripotent or “ground” state, which method comprises small molecule manipulation of one or more intracellular signalling cascades. As demonstrated in the examples, such methods can be used to induce emergence of numerous pluripotent colonies within 1 week.

Furthermore, the inventors have shown that Sox2 transfection is fully dispensable and only three or even two exogenous factors are required to convert NS cells into chimaera-forming iPS cells.

The methods of generating authentic pluripotent stem cells described herein open the door to molecular delineation and dissection of the reprogramming process. Additionally, the rapidity and efficiency of the methods can obviate the need for stable genetic modification of iPS cells (e.g. by retroviral vectors expressing appropriate factors).

Thus a further object is to provide cells which have been completely reprogrammed back into a pluripotent state and which are free from genetic modification. A still further object is to provide an isolated population of cells, which can be maintained in culture and which are truly pluripotent cells, having characteristic of pre-implantation ES cells per se.

Some aspects of the present invention will now be discussed in more detail.

### Factor Expression or Provision

In accordance with one aspect of the invention, transfection of mouse and human tissue cells using plasmids that transiently express reprogramming factors yields reprogrammed pluripotent cells which are not genetically modified compared to the starting cells.

Also in accordance with the invention, EpiSCs and other cells which are undifferentiated but which do not have characterising functional properties of ES cells are converted into cells having additional characterising properties of ES cells by culture in the presence of medium containing a mitogen-activated protein kinase (MAPK) kinase (MEK) inhibitor and a GSK3 inhibitor and an activator of STAT3 (one example of which is the cytokine leukaemia inhibitory factor (LIF)). Particular MAPKs activation of which it may be preferred to inhibit are ERK1 and ERK2 (also called p44 and p42 MAPKs).

The invention accordingly provides a method of reprogramming comprising:—
(a) providing a cell to be reprogrammed;
(b) expressing one or more reprogramming factors in the cell; and
(c) maintaining the cell in culture in the presence of a MEK inhibitor and a STAT3 activator (e.g. LIF), whereby the cell is reprogrammed.

Preferably the reprogramming factors will be transiently expressed in step (b), e.g. from plasmids. As shown in the Examples below, only a single round of the transient infection has been shown to be sufficient to effect reprogramming. In an alternative embodiment reprogramming factors may be introduced by liposomal delivery or microinjection of either mRNAs or proteins prepared in vitro.

Generally the cell may be maintained in culture until extrachromosomal genetic material, if any, introduced during step (b) is lost, thereby providing a reprogrammed cell which is not genetically modified compared to the cell of step (a).

Although the discussion herein is primarily concerned with the application of these inhibitors and LIF to inducing reprogramming in a cell treated with factors which have been expressed transiently e.g. from a plasmid preparation, it will be appreciated that this aspect of the invention applies likewise to cells treated with factors expressed by integration into the genome e.g. introduced via retroviral infection or by factors introduced in the form of mRNAs or proteins. In the Examples below the inventors have employed the piggyBac transposable element system both in integrating and non-integrating (without PBase) methods. This system may lead to reduced silencing of transgenes compared to retroviral vectors. Irrespective of this, it will be understood that in embodiments of the invention integrative vector systems may also be utilised. In such methods the gene encoding the reprogramming factor may optionally be removed or excised following the method.

Thus in one embodiment the invention also provides a method of reprogramming, comprising:—
(a) providing a cell to be reprogrammed;
(b) introducing extrachromosomal genetic material into the cell so that one or more reprogramming factors are transiently expressed in the cell or otherwise provided in the cell; and
(c) maintaining the reprogrammed cell in culture in the presence of MEK inhibitor and STAT3 activator until the extrachromosomal genetic material is lost, whereby a reprogrammed cell is obtained being not genetically modified compared to the cell of step (a).

It will be appreciated by those skilled in the art that the genetic material can be any form which leads to expression e.g. DNA, RNA and so on.

These methods suitably comprise introducing into the cells a plasmid preparation which expresses one or more reprogramming factors in the cell.

In another embodiment the invention also provides a method of reprogramming, comprising:—
(a) providing a cell to be reprogrammed;
(b) introducing or otherwise providing one or more reprogramming factors into the cell; and
(c) maintaining the reprogrammed cell in culture in the presence of MEK inhibitor and STAT3 activator whereby a reprogrammed cell is obtained being not genetically modified compared to the cell of step (a).

A still further method of the invention is a method of reprogramming, comprising:—
(a) providing a cell to be reprogrammed;
(b) introducing into the cell a plasmid preparation which expresses one or more reprogramming factors,
(c) culture in the presence of MEK inhibitor and STAT3 activator wherein the cell is reprogrammed by the reprogramming factors; and
(c) thereby obtaining a reprogrammed cell.

According to the invention, transfection leads to transient expression of the reprogramming factors and, as a result, reprogramming but yielding a population of cells without genetic modification.

Lack of genetic modification in this context will be understood to mean the absence of heterologous nucleic acid sequences (especially those encoding reprogramming factors) stably introduced in the genome of the cell. By “heterologous” is meant that the nucleic acid in question has been introduced into said cell or an ancestor thereof, using genetic engineering. A heterologous nucleic acid may normally be absent from cells of that type (e.g. retroviral sequence) or may be additional to an endogenous gene of the cell (e.g. an additional copy of a reprogramming factor, where the endogenous copy has been inactivated) but in each case the heterologous nucleic acid is introduced by human intervention.

The nature of the transfection may be that extended culture of the reprogrammed cells results in loss of the transfection agent. Confirmation that cells are obtained with no genetic modification can be achieved by screening clones of the cells and analyzing the DNA, for example using PCR or Southern Blot methodology—using this approach we have confirmed absence of genetic modification in cells obtained by these methods.

Expression of the reprogramming factors is suitably achieved using genetic material introduced into the cells and containing coding sequences for the reprogramming factors operatively linked to promoters; preferably, plasmids are used. The promoters direct expression of the reprogramming factors and, generally, a constitutive promoter is suitable—a CAG promoter was used in some of the Examples herein—but the choice of promoter is not critical provided the reprogramming factors are expressed in the cells. Other suitable promoters include PKG and CMVE. The genetic material, such as the plasmids, further preferably does not replicate and has a very low integration efficiently, which can be further reduced e.g. by using circular rather than linear plasmids.

Plasmids are preferably introduced by using nucleofection which is an established procedure and known to be efficient. Other chemical and electrical methods are known and are also efficient, including electroporation and lipofection. Different transfection methods and protocols are available for different cells, all well known in the art. Generally, it is believed that the choice of plasmid and promoter and transfection route is not critical to the invention. The plasmid preparation comprises one or more plasmids which express in the cell the one or more reprogramming factors. There may be one plasmid for each factor or a plasmid may express more than one or all factors.

Typically, the time period of expression of the reprogramming factors in accordance with the invention is that expression peaks from 12 to 96 hours, more typically from 24 to 72 hours after transfection and thereafter declines. In specific examples, expression peaks at about 48 hours after transfection. It is surprising that reprogramming can be achieved with this time period, as others have taught that several weeks of expression using retroviruses is needed and that shorter periods lead to incomplete reprogramming.

### Preferred Reprogramming Factors and Combinations Thereof

Reference to one or more reprogramming factors is reference to one or to more than one or to a combination of factors which when expressed in the cell result in it being reprogrammed. Generally, a combination of factors is used and the reprogramming factors include one or more transcription factors.

Preferably a combination of two, three or more factors may be used. Examples of suitable reprogramming factors are Oct3/4, Sox2, Klf4, Nanog, LIN28 and c-Myc. As described below, in certain embodiments Sox2 may be omitted. As shown below, two factors may be employed e.g. Oct4 plus cMyc, or Oct4 plus klf4. Indeed in embodiments of the invention a single factor i.e. Oct4 may be utilised. As shown in the examples below, Nanog, Klf4 and Klf2 all have particular utility in reprogramming EpiSCs.

Others are selected from the family members of these specific factors and sequence data for these are available in established databases, for example the Ensembl Gene Browser database. Yamanaka himself who worked on retrovirus-based methods has tested different family members of the first transcription factors he used and found family members to be suitable too; it is to be expected that further factors will be found that can be used for reprogramming and these are embraced by the present invention. In specific examples described below, a combination of the same four factors as used by Yamanaka has been used to achieve reprogramming. Groups other than Yamanaka used different combinations of factors, and these combinations are included in the present invention.

### Preferred Cells for Reprogramming

The cell to be reprogrammed is preferably mammalian, in particular mouse, rat, primate, ovine, bovine, porcine or human. In examples we have to date used mouse and human cells. Preferably, the cell is a human cell. However application of the present invention to avian cells is also encompassed.

The cell to be reprogrammed can be a somatic stem cell by which is meant an undifferentiated cell found in the body in a differentiated tissue that can renew itself and differentiate (with certain limitations) to give rise to all the specialized cell types of the tissue from which it originated within the body. Preferably, the cell may also be a neural cell, in particular a neural stem cell—in examples we have used both mouse and human neural stem cells. Mouse and human brain derived neural stem cells have the potency to differentiate into neurons, atrocytes and oligodendrocytes, and can be stably propagated as undifferentiated clonal populations in adherent serum-free culture (Conti, 2005; Pollard, 2006; Sun, 2008). However other examples may include bone marrow stromal cells. Preferably, the cell is a human neural stem cell. More generally, it is preferred that the cell to be reprogrammed is a diploid cell which may be ‘wild-type’ or non-transformed cell. In other embodiments it may be a transformed (tumour) cell. Preferably, the starting cell is obtained from a homogenous cell population. Neural stem cells, especially the mouse and human neural stem cells which have successfully been used to date, are good examples of these but other cells showing these properties are also suitable for the methods of the invention.

As described below, the use of the inhibitors compositions as descrined herein, may also have utility with EpiSCs, or other human embryo derived or reprogrammed stem cells which are related to EpiSCs. Where aspects or embodiments of the invention are described below with respect to “intermediate” or “partially” reprogrammed cells, it will be understood that the invention may be likewise applied to EpiSCs, or other human embryo derived stem cells which are related to EpiSCs.

Cells may be obtained from an individual by standard techniques, for example by biopsy for skin cells. Cells may preferably be obtained from an adult.

Cells may be obtained from pre-existing cell lines without need for biopsy. For example the invention is applicable to pre-existing embryonic stem cell lines.

The cell to be reprogrammed may also be a cell which already expresses one the reprogramming factors. The invention thus forcibly expresses the remaining of the reprogramming factors. For example, the cell may already express Oct 3/4, Klf4 or Sox2.

Some of all of these factors may already have been expressed from heterologous nucleic acid introduced at an earlier stage of the method. The methods of the invention may optionally comprise “further introducing” or “reintroducing” nucleic acid encoding a reprogramming factor e.g. Klf4 or Klf2, where desired. This is in addition to the use of inhibitors of intracellular signalling cascades as described below.

After introduction of genetic material to express the reprogramming factors, the cell is preferably maintained in culture to allow reprogramming of the cell and growth of the cell. The cell is preferably maintained in medium containing LIF or an alternative agonist of the LIF receptor, such as IL-6 and soluble IL-6 receptor. For human cells, human LIF is preferably used.

### Uses of Inhibitors of Intracellular Signalling Cascades

In certain aspects of the invention, during at least part of the reprogramming process, the cell is also preferably maintained in the presence of a one or more kinase inhibitors which inhibits a kinase responsible for an intracellular signalling cascade e.g. in the presence of a MEK inhibitor, a GSK3 inhibitor or both a MEK inhibitor and a GSK3 inhibitor or inhibitor of other kinases within these same cascades.

As shown in the Examples below, the final transition from EpiSC or pre-IPS cell may be efficiently induced by blockade of ERK1 or ERK2 signaling (a MEK inhibitor) in conjunction with stimulation by LIF. GSK3 inhibition consolidates this process.

In preferred embodiments, in addition to treatment with one or more reprogramming factors (e.g. two, three or more, as described herein) the cells are treated with a medium comprising:

(i) a MEK inhibitor or (preferably) both a MEK inhibitor and a GSK3 inhibitor,

### (ii) LIF,

in order to induce reprogramming into a fully reprogrammed, pluripotent cell.

Use of a medium as described above for this purpose (to induce reprogramming into a fully reprogrammed, pluripotent cell) forms another aspect of the present invention.

Use of a MEK inhibitor and a GSK3 inhibitor and LIF in the preparation of an agent to induce reprogramming of a cell into a fully reprogrammed, pluripotent cell forms another aspect of the present invention.

As described below, the present inventors have demonstrated that NS cells and EpiScs can be triggered to undergo conversion to full induced pluripotency more rapidly, at higher frequency, and with fewer retroviral insertions than fibroblasts treated in parallel. Thus the present inventors have found that using “2i” medium with LIF, NS cell reprogramming required only 1-2 integrations of each transgene. Along with the transient expression data described herein, this supports the conclusion that a mutagenic requirement is not obligatory for reprogramming (Aoi et al., 2008). In any case, the low copy number greatly increases the feasibility of excising transgenes, for example by site specific recombination, to generate genetically unmodified iPS cells.

It will be understood that in these aspects and embodiments, other kinase inhibitors which inhibit a kinase responsible for an intracellular signalling component of the same cascades (e.g. ERK1 or ERK2 cascade) may be substituted where desired for the MEK inhibitor or GSK3 inhibitor. This may include inhibition of an upstream stimulus of the MAPK pathway, in particular through the FGF receptor (Ying, Nature, 2008). Likewise the LIF may be substituted where desired for other activators of Stat3 or gp130

In all of these aspects, starting cells may be characterized by, for example (and without limitation) the incomplete expression of pluripotency associated genes; non-responsiveness to LIF; retention of epigenetic silencing of the X chromosome; inability to yield chimaeras.

In all of these aspects, the conversion to pluripotency can be assessed as described elsewhere herein, for example (and without limitation) expression of Nanog, Rex1 and endogenous Oct4 and Klf4 mRNAs at levels similar to ES cells; immunofluorescent staining revealed the expected nuclear localisation of Nanog; reactivation of a silenced X chromosome by absence of an me3H3K27 nuclear body; ability to colonize chimaeras i.e. competence for somatic and germline chimaerism (in non-human animals).

Inhibitors may be provided or obtained by those skilled in the art by conventional means or from conventional sources, and such inhibitors per se are not part of the present invention (see also WO2007113505).

Reference to GSK3 inhibition refers to inhibition of one or more GSK3 enzymes. The family of GSK3 enzymes is well-known and a number of variants have been described (see e.g. Schaffer et al.; Gene 2003; 302(1-2): 73-81). In specific embodiments GSK3-6 is inhibited. GSK3-a inhibitors are also suitable, and in general inhibitors for use in the invention inhibit both. A wide range of GSK3 inhibitors are known, by way of example, the inhibitors CHIR 98014, CHIR 99021, AR-AO144-18, TDZD-8, SB216763 and SB415286. Other inhibitors are known and useful in the invention. In addition, the structure of the active site of GSK3-β has been characterised and key residues that interact with specific and non-specific inhibitors have been identified (Bertrand et al.; J Mol Biol. 2003; 333(2): 393-407). This structural characterisation allows additional GSK inhibitors to be readily identified.

The inhibitors used herein are preferably specific for the kinase to be targeted. The inhibitors of certain embodiments are specific for GSK3-β and GSK3-α, substantially do not inhibit erk2 and substantially do not inhibit cdc2. Preferably the inhibitors have at least 100 fold, more preferably at least 200 fold, very preferably at least 400 fold selectivity for human GSK3 over mouse erk2 and/or human cdc2, measured as ratio of IC₅₀ values; here, reference to GSK3 IC₅₀ values refers to the mean values for human GSK3-β and GSK3-α. Good results have been obtained with CHIR 99021 which is specific for GSK3. Examples of GSK3 inhibitors are described in Bennett C, et al, J. Biol. Chem., vol. 277, no. 34, Aug. 23, 2002, pp 30998-31004 and in Ring D B, et al, Diabetes, vol. 52, March 2003, pp 588-595. Suitable concentrations for use of CHIR 99021 are in the range 0.01 to 100, preferably 0.1 to 20, more preferably 0.3 to 10 micromolar.

Reference to a MEK inhibitor herein refers to MEK inhibitors in general. Thus, reference to a MEK inhibitor refers to any inhibitor a member of the MEK family of protein kinases, including MEK1, MEK2 and MEK5. Reference is also made to MEK1, MEK2 and MEK5 inhibitors. Examples of suitable MEK inhibitors, already known in the art, include the MEK1 inhibitors PD184352 and PD98059, inhibitors of MEK1 and MEK2 U0126 and SL327, and those discussed in Davies et al (2000) (Davies S P, Reddy H, Caivano M, Cohen P. Specificity and mechanism of action of some commonly used protein kinase inhibitors. Biochem J. 351, 95-105). In particular, PD184352 and PD0325901 have been found to have a high degree of specificity and potency when compared to other known MEK inhibitors (Bain, Biochem J. 2007). Other MEK inhibitors and classes of MEK inhibitors are described in Zhang et al. (2000) Bioorganic & Medicinal Chemistry Letters; 10:2825-2828. A preferred inhibitor combination is PD0325901 plus CHIR99021 used in the Examples below.

Inhibition of MEKs can also be conveniently achieved using RNA-mediated interference (RNAi). Typically, a double-stranded RNA molecule complementary to all or part of a MEK gene is introduced into pluripotent cells, thus promoting specific degradation of MEK-encoding mRNA molecules. This post-transcriptional mechanism results in reduced or abolished expression of the targeted MEK gene. Suitable techniques and protocols for achieving MEK inhibition using RNAi are known.

A number of assays for identifying kinase inhibitors, including GSK3 inhibitors and MEK inhibitors, are known. For example, Davies et al (2000) describe kinase assays in which a kinase is incubated in the presence of a peptide substrate and radiolabelled ATP. Phosphorylation of the substrate by the kinase results in incorporation of the label into the substrate. Aliquots of each reaction are immobilized on phosphocellulose paper and washed in phosphoric acid to remove free ATP. The activity of the substrate following incubation is then measured and provides an indication of kinase activity. The relative kinase activity in the presence and absence of candidate kinase inhibitors can be readily determined using such an assay. Downey et al. (1996) J Biol Chem.; 271(35): 21005-21011 also describes assays for kinase activity which can be used to identify kinase inhibitors.

### Preferred Culture Conditions

The starting cell and the end, reprogrammed cell generally have differing requirements for culture medium and conditions. To allow for this whilst also allowing that reprogramming of the cell is taking place, it is usual to carry out at least an initial stage of culture, after introduction of the reprogramming factors, in the presence of medium and under culture conditions known to be suitable for growth of the starting cell. This is followed by a subsequent period of culture in the presence of medium and under conditions known to be suitable for pluripotent cells—typically in serum on feeders or in the presence of LIF (optionally supplemented by BMP); alternatively in the presence of a MEK inhibitor or a GSK3 inhibitor or both, and this alternative step can also come after the use of serum and LIF.

The initial stage of culture is preferably for a period of up to 6 days, more preferably up to 4 days and in particular embodiments, described below for not more than 3 days. The subsequent stage of culture is suitably for a period of at least 14 days, preferably at least 21 days and can be for a period of up to 70 days, preferably up to 56 days. In a specific embodiment described below, used to generate reprogrammed human cells, the initial stage of culture was for a period of about 3 days and the subsequent stage was for about 6 weeks, followed by culture in the presence of both a MEK inhibitor and a GSK3 inhibitor.

Thus, in a method of the invention, a neural cell is reprogrammed by the method, comprising:—
(a) providing a neural cell to be reprogrammed;
(b) introducing into the cell a plasmid preparation which expresses one or more reprogramming factors;
(c) culturing the cell in the presence of neural cell growth factors, optionally EGF and FGF;
(d) culturing the cell from (c) in the presence of serum and optionally LIF;
(e) culturing the cell from (d) in the presence of a MEK inhibitor (and preferably a GSK3 inhibitor) and LIF or some other Stat3 stimulator; and
(f) thereby obtaining a reprogrammed cell

In this method, the EGF and FGF are examples of factors which promote growth and survival of the neural cell immediately after transfection with the plasmids. Other suitable NS media include NSA supplemented with EGF and FGF and N2B27 supplemented with FGF and EGF. LIF is an example of an activator of gp130 signalling, another being IL-6 in combination with soluble IL-6 receptor, and promotes growth and survival of the cell as it is in the process of being reprogrammed and the combination of a MEK inhibitor and a GSK3 inhibitor promote final conversion of the cell into a pluripotent cell. During reprogramming, cells are hence preferably cultured in the presence of LIF; using LIF helps with human and mouse reprogrammed cell capture and improves cell survival and clonogenicity.

In a specific embodiment of this method, illustrating the invention, and an example of which is described in more detail below, human NS (neural stem) cells are nucleofected with plasmids expressing reprogramming factors. These cells are cultured in medium supplemented with EGF and FGF. The cells are then cultured in medium supplemented with serum replacement and LIF. A cell or cells from emerging colonies is isolated and cultured on feeders in the presence of serum replacement and LIF. Subsequently the medium is changed to a medium supplemented with a MEK inhibitor and a GSK3 inhibitor in addition to LIF. ES-like colonies are obtained, believed to be truly reprogrammed pluripotent cells. The cell characteristics and morphology are observed to be the same as for the corresponding mouse reprogrammed cells and as for known mouse ES cells. Suitable feeders include primary or immortalized fibroblast lines, typically inactivated so they do not overgrow the growth of the cells being reprogrammed.

### Reporter Systems

In a further embodiment of the invention, described in an example below in more detail, NS cells are transfected with a plasmid preparation expressing reprogramming factors. The transfected cells include a reporter system enabling identification of pluripotent cells—such as an Oct4-based reporter system whereby Oct-4 expressing cells can either be selected in culture or express a gene such as a fluorescent gene, enabling easy identification of cells which express Oct-4. After transfection, the cells are grown in media containing serum and LIF (at this stage in the examples, FACS analysis indicated that no cells were expressing Oct-4). The cells are passaged, typically 3-4 times and then transferred to media containing a MEK inhibitor and a GSK3 inhibitor. After culture in this media cells are obtained which express the reporter/marker, indicating that the cells are pluripotent (e.g. that Oct4 is now being expressed) and the cells have been reprogrammed. However, as noted below, it will be understood by those skilled in the art that neither Oct4 reporter activation (nor morphology) are definitive markers for pluripotent identity, and confirmation of that identity via chimaera formation or reactivation of a silenced X chromosome will therefore generally be required.

### Preferred Embodiments of the Invention

### Expression Systems, Inhibitors, and Reduced Numbers of Reprogamming Factors

The plasmid preparation used in the methods can contain many types of plasmids—one for each reprogramming factor—or a reduced number of plasmids, even a single plasmid, whereby several factors are expressed from the same plasmid. A further option is to prepare one or more synthetic constructs which contain between them the reprogramming factors linked to promoters so that after introduction in the cell the factors are expressed. An option is to engineer a recombinant DNA molecule which would express the factors but which would not integrate.

The invention further preferably uses non-replicating DNA to express the factors. A small level of replication can in some embodiments be tolerated and might even be useful in reprogramming cells which require more time to be reprogrammed. With NS cells, both mouse and human, the time period of reprogramming is quick but with other cells more time may be needed.

In accordance with the invention, reprogramming may be achieved by transient expression of the one or more reprogramming factors. This expression is transient in that it does not have to be switched off or down regulated by intervention, whether genetic intervention, intervention by change of culture additives or otherwise by an external operator. Instead, the expression is temporary and stops spontaneously after a period of time. In embodiments of the invention, one or more replication factors are expressed on plasmids, and it is found that the cell to be reprogrammed is readily transfected by the plasmids so that expression occurs as desired but that, in addition, after maintenance of the cell the plasmids are spontaneously lost without any genetic modification or chromosomal integration having taken place, the resultant reprogrammed cell not containing the plasmids or the ghosts or residue of plasmids. Plasmid is lost e.g. by its destruction in the host cell and absence of plasmid replication progressively diluting out the plasmid as cells grow in culture.

It is surprisingly found that although the expression is transient and automatically stops, there is nevertheless expression at sufficient level and for a sufficient period of time to obtain partly reprogrammed cells, which cells can be converted to the fully pluripotent state in accordance with the invention, preferably by the use of culture medium comprising a MEK inhibitor, a GSK3 inhibitor or both a MEK inhibitor and a GSK3 inhibitor which together with LIF enables selection through culture conditions, rather than through any genetic or reporter method for those reprogrammed cells.

In embodiments of the invention, a method of reprogramming cells comprises culturing cells in a MEK inhibitor or preferably both a MEK inhibitor and a GSK3 inhibitor in the presence of an activator of Stat3. Thus, following the invention, partially reprogrammed cells, for example cells obtained using the retroviral-based methods of the art or the transient expression based method of the invention, are converted to pluripotent cells by culture in the presence of a MEK inhibitor, or preferably in the presence of both a MEK inhibitor and an inhibitor of GSK3.

The invention thus also provides a method of converting a partially reprogrammed cell into a fully reprogrammed, pluripotent cell, comprising maintaining the partially reprogrammed cell in culture medium containing an inhibitor as described above e.g. a MEK inhibitor, or both a MEK inhibitor and a GSK 3 inhibitor and an activator of Stat3.

The partially reprogrammed cell may be obtained by expression or other provision in a somatic cell of one or more reprogramming factors, e.g. retrovirally or on plasmids, or from introduced mRNAs or proteins.

As per the above, a method of activating an X chromosome in a pluripotent cell having 2 X chromosomes, one of which is inactive, is still further provided by the invention, the method comprising culturing the pluripotent cell in medium containing a MEK inhibitor, a GSK 3 inhibitor or both a MEK inhibitor and a GSK 3 inhibitor, whereby a pluripotent cell is obtained having 2 active X chromosomes. In this method, the cell is preferably a human cell.

So-called 1i media (containing a MEK inhibitor and preferably LIF) or 2i media (containing a MEK inhibitor and a GSK3 inhibitor) or 31 media (containing a MEK inhibitor and a GSK inhibitor and an FGF receptor inhibitor as described by Ying, 2008) is thus used in the invention to convert pluripotent cells which do not have the characteristics of true ES cells into cells which do, or to complete the conversion of somatic cells into ES-like pluripotent cells. This conversion has been carried out by the inventors on cells in which the initial reprogramming has been carried out using both a known protocol (e.g. Yamanaka's retroviral-based method) and a transient plasmid expression method of the invention.

The present invention thus relates to a new use for the inhibitors described above i.e. for actual induction of the conversion of incompletely reprogrammed cells which do not have the characteristics of true ES cells into cells which do, or to complete the conversion of somatic cells into ES-like pluripotent cells. The phenotypes of these cells are discussed above.

The present inventors have found that both mouse and human NS cells can be converted without the need for exogenous Sox2 or c-Myc, although omission of c-Myc incurs delayed kinetics and lower efficiency. This is consistent with the findings of Kim et al. 2008. Furthermore, the inventors have found that NS cells are converted significantly more efficiently without exogenous Sox2.

The invention further provides a method of reprogramming a cell, comprising: (a) providing a cell to be reprogrammed; (b) introducing into the cell heterologous nucleic acid encoding one or more (e.g. 1, 2, 3, 4 or 5) reprogramming factors, wherein the cell is reprogrammed into a reprogrammed cell by expression of the reprogramming factors; and (c) thereby obtaining a reprogrammed cell, wherein the reprogramming factors are selected from Oct3/4, Klf4, c-Myc, Nanog and LIN28, with the proviso that the heterologous nucleic acid does not encode Sox2.

As noted above, with NS cells, the time period of primary reprogramming is quick (potentially a single round of transient expression, even in the absence of inhibitors). The use of NS cells (such as rodent NS cells) for example with these reduced combinations of reprogramming factors, optionally with the inhibitors described above is a preferred aspect of the invention. For example the inventors have shown that transduction with Sox2 and c-Myc is dispensable, and Oct4 and Klf4 are sufficient to convert NS cells into chimaera-forming iPS cells.

Thus optionally the heterologous nucleic acid may encode one or more (e.g. 1, 2, 3 or 4) reprogramming factors selected from Oct3/4, Klf4, Nanog and LIN28, with the proviso that the heterologous nucleic acid does not encode Sox2 or c-Myc.

Preferably the reprogramming factors consist of Oct4 and Klf4.

In other embodiments the reprogramming factors consist of Nanog and Klf4.

In other embodiments the reprogramming factors consist of Nanog or Klf4 alone.

In other embodiments Klf4 as described in relation to any of the embodiments herein, may be substituted by Klf2.

The present invention in addition relates to and provides new pluripotent cells.

The invention provides an isolated pluripotent stem cell obtained by a method according to the invention.

The invention provides an isolated reprogrammed pluripotent stem cell, characterised in that:—

(i) it can be maintained in a pluripotent state in culture medium containing a MEK inhibitor (plus optionally a GSK3 inhibitor) and a STAT3 activator; and
(ii) it does not require FGF/MAPK signalling in order to self-renew.

The cells are preferably further characterized, if female, by having two active X chromosomes.

Particularly preferred embodiments of the invention, provide pluripotent cells which are still further characterized by one or more or all of the following properties:—

(i) they differentiate upon exposure to FGF;
(ii) they grow in medium containing LIF and BMP;
(iii) they grow in 2i medium;
(iv) they express Rex1;
(v) they express Nanog; and
(vi) they express markers specific for pre-implantation pluripotent cells.

Cells, particularly human cells, of any of the aspects of the present invention may be also be provided as populations of cells (or isolated populations of cells) in which at least 1, 2, 3, 4, 5, 6, 7, 8, 9, 10, 20, 30, 40, 50, 60, 70, 80, 90, or 100% of said cells have the aforementioned characteristics. Thus the methods and processes of the invention may be used to provide isolated or enriched populations pluripotent stem cells having desirable properties.

The invention can deliver advantageous and surprising results. According to the prior art, the dynamics of reprogramming are slow and take many weeks to complete, as reported for example by Meissner et al. Plasmid-based systems are known to offer only limited time periods of expression, no replication in the host and dilution out of plasmid from growing cells over time, and would thus not be expected to work; plasmid based expression would very typically peak after 2-3 days and then decline.

In specific embodiments of the invention, human reprogrammed cells are obtained which are pluripotent and have the following properties, contrasted below with the properties of known human cells:—

| | Known alleged | Human pluripotent |
|---|---|---|
| | pluripotent “human | cells obtained |
| | ES cells” and human | according to the |
| Feature | “iPS” cells | invention |
| Need for FGF? | Need FGF | Don't need FGF, |
| | | but differentiate in |
| | | the presence of FGF |
| Response to 2i media | Cells die | Cells self-renew |
| Response to LIF | None | Enhanced |
| | | self-renewal |
| Morphology | ES-like | ES-like |
| Activation of X | One silent | Both X chromosomes |
| chromosome in | X chromosome | activated |
| XX cells? | | |
| Markers of epistem cell | Present | Absent |
| phenotype | | |
| Exclusive markers of ES | Absent | Present |
| cell phenotype | | |

In reprogramming cells in accordance with the present invention, it has been possible successfully to reprogram mouse and human cells substantially without the production of secondary cell derivatives, the cell population obtained being a substantially homogenous population of pluripotent cells. It has been possible to avoid the use of feeder cells.

Importantly no genetic selection techniques were necessary to identify the programmed cells and there was similarly no need for expression of reporter genes.

Following the methods described herein, it is possible to obtain pluripotent cells, whether of mouse or human or other mammalian origin, which are truly reprogrammed back into a reprogrammed state and which are not genetically modified.

In other reprogramming methods of the present invention EpiSCs have been converted to ground state using single transgenes. For example using an integrated Nanog cDNA transgene. Thus Nanog (optionally with Klf2 of Klf4) can reprogramme EpiSCs. The inventors further showed transient expression of Nanog and Klf4 was also sufficient to reprogramme EpiSCs.

Thus in other aspects the invention provides methods of reprogramming an EpiSCs (e.g. to ground state pluripotency) comprising: (a) providing an EpiSC cell to be reprogrammed; (b) introducing into the cell a nucleic acid or protein preparation which expresses or provides one or more of reprogramming factors discussed above. Nucleic acid may be expressed transiently or integrated. Cells obtainable by use of the method (e.g. mouse or human cells) are also provided, as are populations of these cells.

Preferably at least Nanog is expressed, optionally with Klf4 or Klf2. The cells will generally be cultured in medium comprising a Stat3 activating cytokine such as LIF. These methods may be performed independently of the use of the inhibitors described hereinbefore.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

In particular WO 2007/069666 (also published as EP 1 970 446) describes the accession numbers of a large number of factors through which a differentiated human cell is said to be converted into a pluripotent state. The disclosure of this publication, particular with reference to the sequence accession numbers of these genes, is specifically incorporated herein by cross reference.

WO2007113505 relates to various serum-free culture media comprising a MEK inhibitor, a GSK3 inhibitor and, optionally, an antagonist of an FGF receptor which may be used to maintain pluripotent in a self-renewing state. The disclosure of this publication, particular with reference to sources of inhibitors, is also specifically incorporated herein by cross reference.

### EXAMPLES

The invention is described in specific embodiments with reference to the accompanying drawings, in which:—

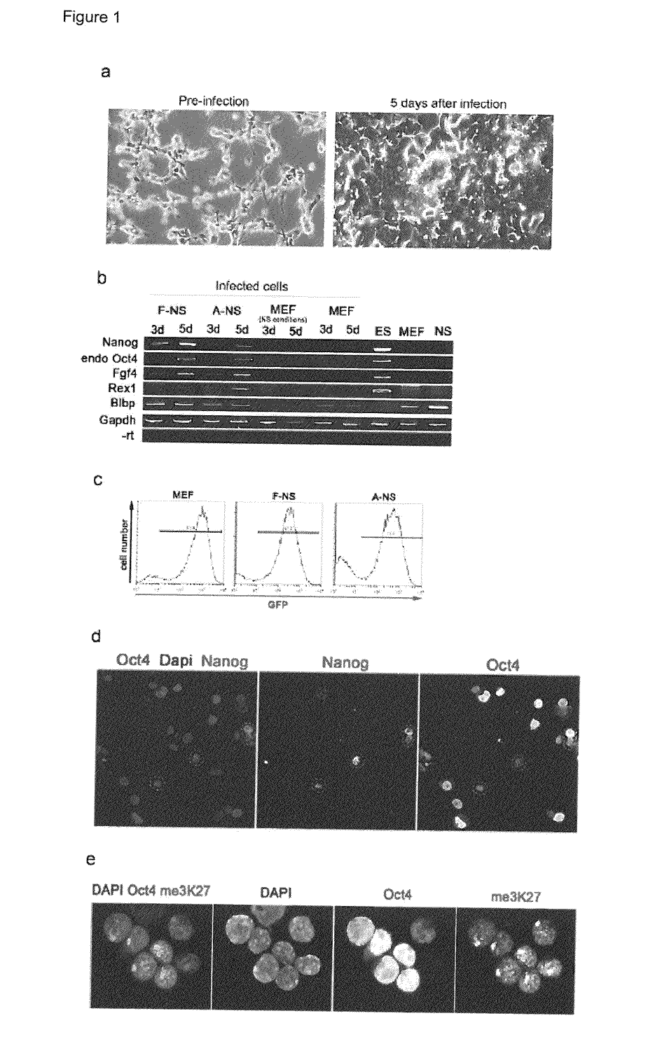

FIG. 1: NS cells undergo rapid but incomplete conversion towards a pluripotent phenotype (a), Phase contrast image of NS cells in standard culture conditions (left) and 5 days after infection with the four factors (right). (b), RT-PCR analyses for Nanog, endogenous (endo) Oct4, Fgf4, Rex1 and Blbp in infected foetal (fNS) and adult (aNS) cells 3 and 5 days after infection. MEF infections under both MEF and NS cell culture conditions were analysed in parallel; (c) Flow cytometry shows comparable infection and expression of control GFP retrovirus in MEFs, fNS and aNS cells. (d) and (e), Immuonofluorescence staining for Oct4 and Nanog—(d) or trimethyl H3K27 (me3K27) (e) 5 days after infection of NS cells with the four factors. Dashed circles outline Nanog positive cells. Arrowheads indicate the nuclear body diagnostic of the inactive X chromosome.

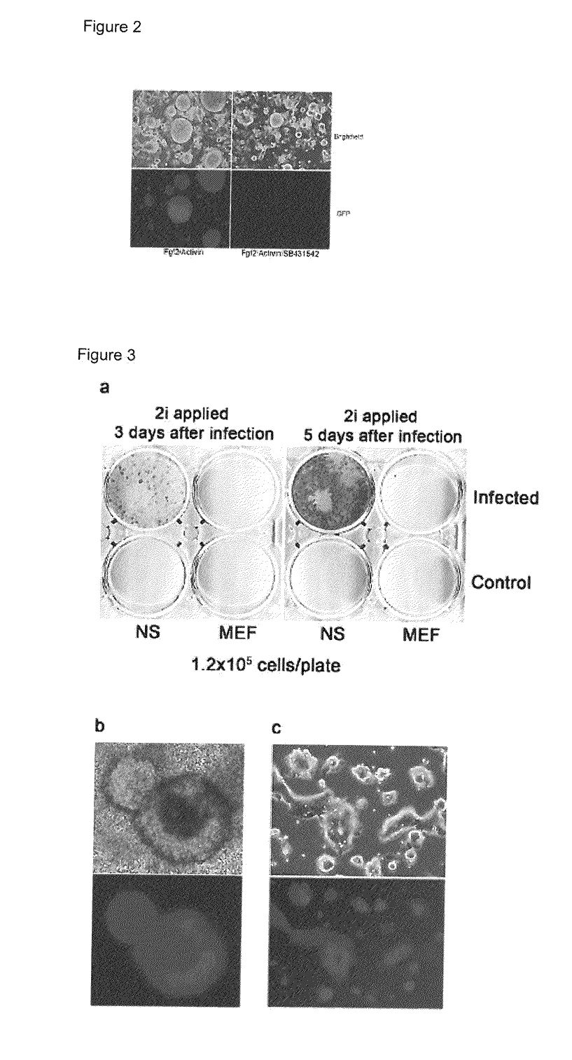

FIG. 2: Oct4-GFP expressing I-iPS cells do not acquire full pluripotent status. Culture of FACS purified GFP expressing incomplete (I) iPS cells in Fgf2 and activin (left) and subsequently on addition of activin receptor inhibitor SB431542 (right).

FIG. 3: MEK and GSK3 inhibitors (2i) promote reprogramming. Plates containing control and infected a NS cells and MEFs were cultured in 2i media from either day 3 or day 5 after infection and stained for alkaline phosphatase 10 days later.

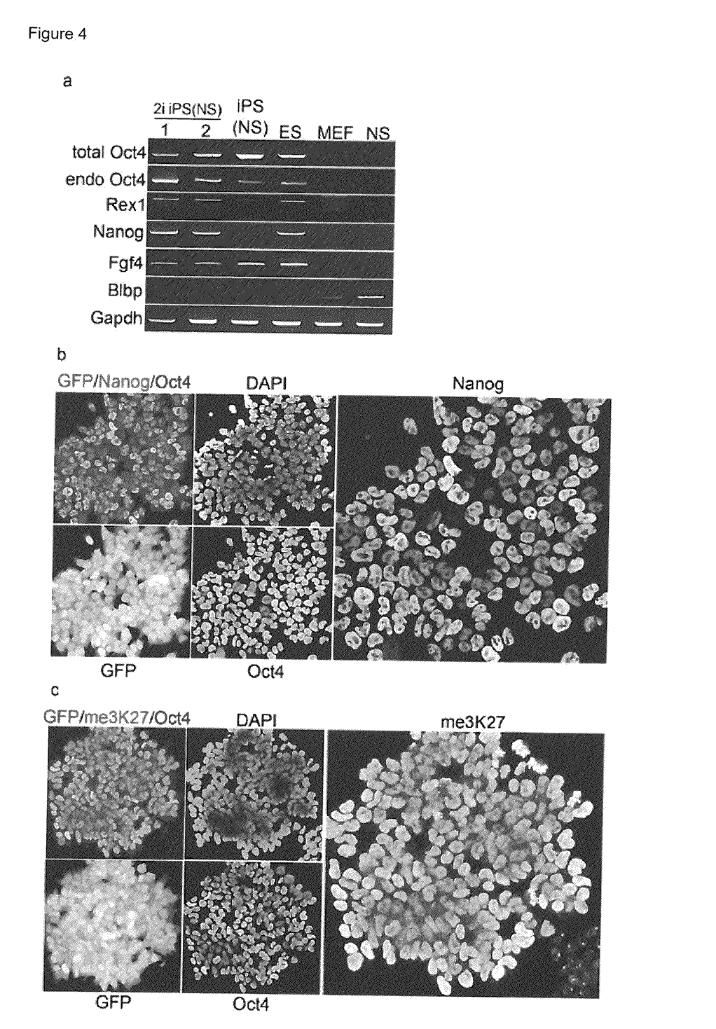

FIG. 4: 2i-iPS cells are fully pluripotent a, RT-PCR analysis for total Oct4, endogenous (endo) Oct4, Rex1, Nanog, Fgf4 and Blbp in 2i-iPS cells generated from NS cells. (b) and (c), Immunofluorescence staining of 2i-iPS cells for Oct4 and Nanog (b) or trimethyl H3K27 (me3K27) (c).

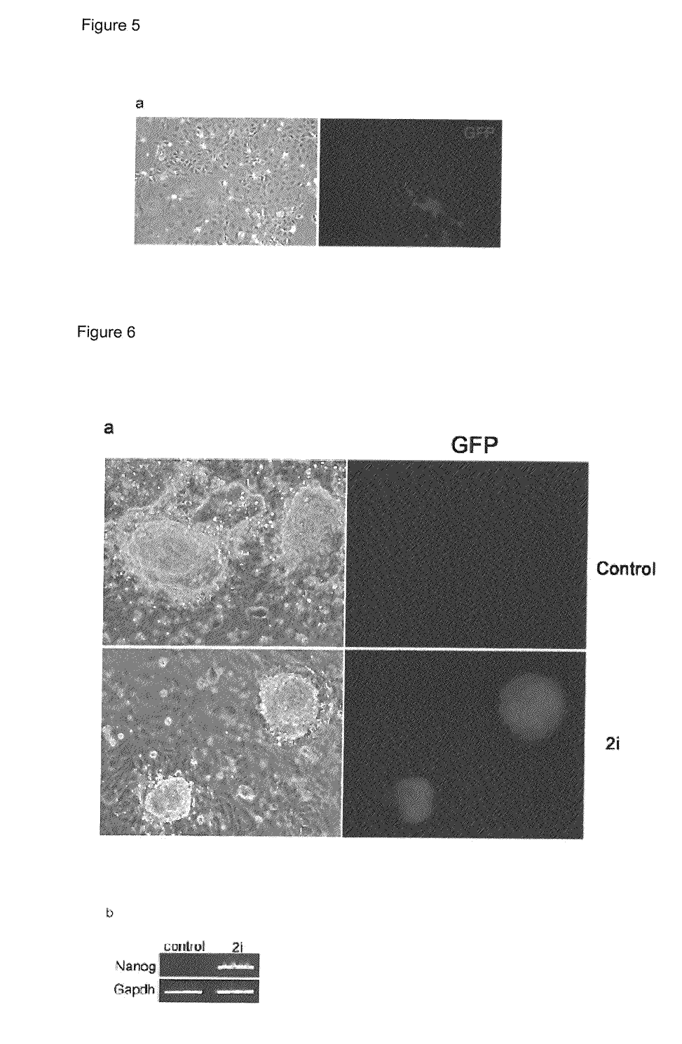

FIG. 5: 2i-iPS cells differentiate or self-renew in a similar manner to ES cells. The Figure shows differentiation of 2i-iPS cells in serum without feeders and LIF.

FIG. 6: 2i promotes nuclear reprogramming; (a), Representative images of colonies generated from I-iPS cells, expanded over 5 passages, plated at clonal density in duplicate wells and cultured in ES cell medium (control) or in 2i medium from day 6 (2i). Images were collected 13 days after plating. (b) RT-PCR analyses for Nanog of I-iPS cells expanded in either control or 2i medium.

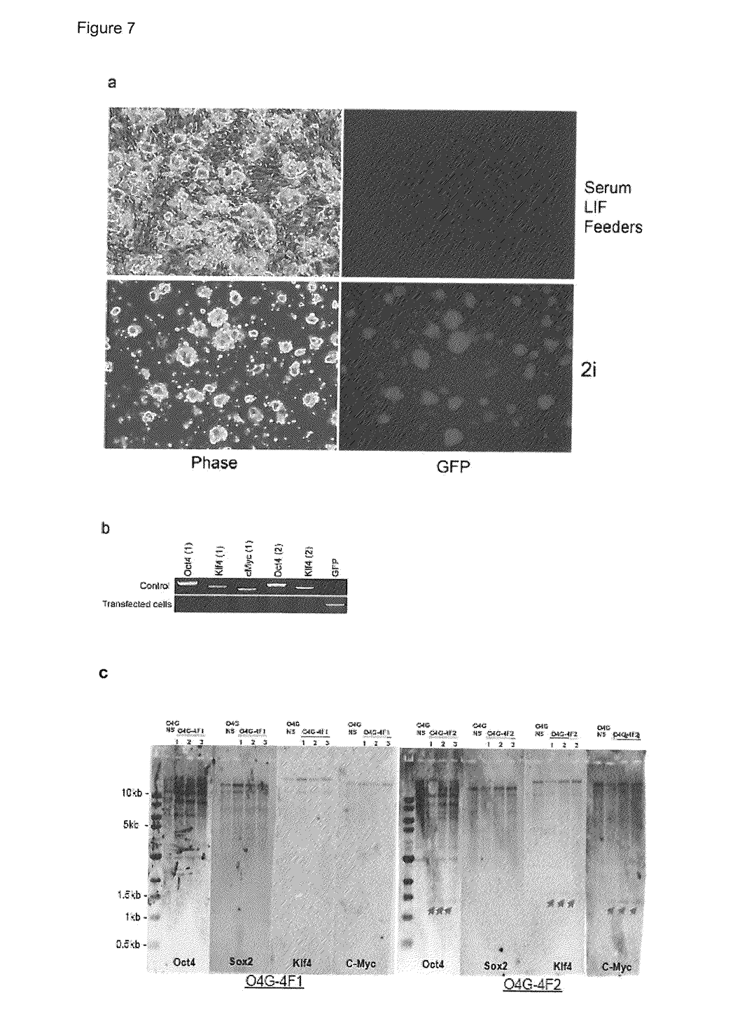

FIG. 7: NS cells convert into pluripotent cells without genomic incorporation of reprogramming factors. The morphology of expanded cells resembles wt ES cells cultured in 2i (FIG. 6a). Results of genomic PCR and southern blot analysis to confirm that the cells did not incorporate the plasmids (FIGS. 6b and 6c).

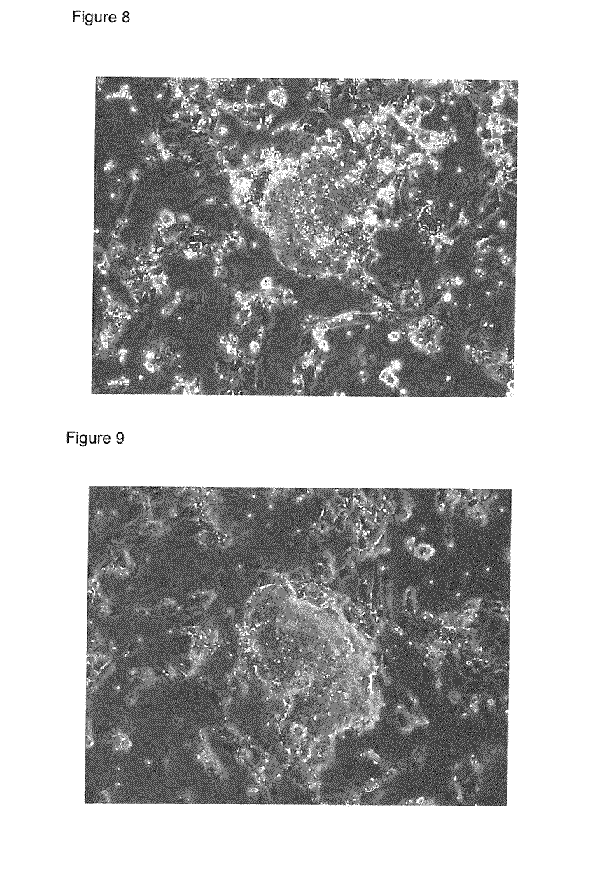

FIG. 8: A population of human pluripotent cells obtained by reprogramming human NS cells.

FIG. 9: A second population of human pluripotent cells obtained by reprogramming human NS cells.

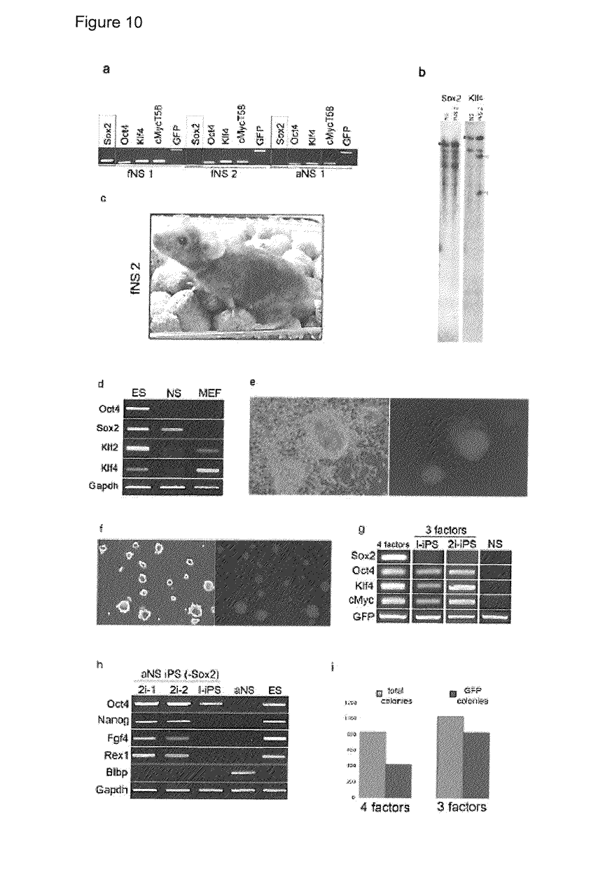

FIG. 10: Sox2 is dispensable for NS cell conversion to pluripotency a, Genomic PCR analysis for retroviral integration in 2i-iPS clones generated from NS cells that were infected with the four factors. b, Southern blot analysis for Sox2 and Klf4 retroviral integrations in INS 2 cells. Arrows indicate retroviral integrations. Red dots indicate endogenous Sox2 and Klf4 locus respectively. c, Chimera generated from injection of fNS 2 2i-iPS cells into C57BL/6 host blastocyst. d, RT-PCR analyses for Oct4, Sox2, Klf4 and Klf2 expression. e-h, Analysis of aNS cells infected with Oct4, Klf4 and cMyc (3 factors). Example of emerging 2i-iPS cell colonies (e). Established 2iiPS cell line (f). Genomic PCR analysis for retroviral integration in incomplete (l) iPS and 2i-iPS cells (g). RT-PCR analysis of pluripotency markers in 2i-iPS cells and I-iPS cells (h). i, Comparison of the reprogramming efficiency of aNS cells infected with either four factors or three factors. 8×105 aNS cells were infected, medium switched to 2i at day 5 and counts performed 5 days later.

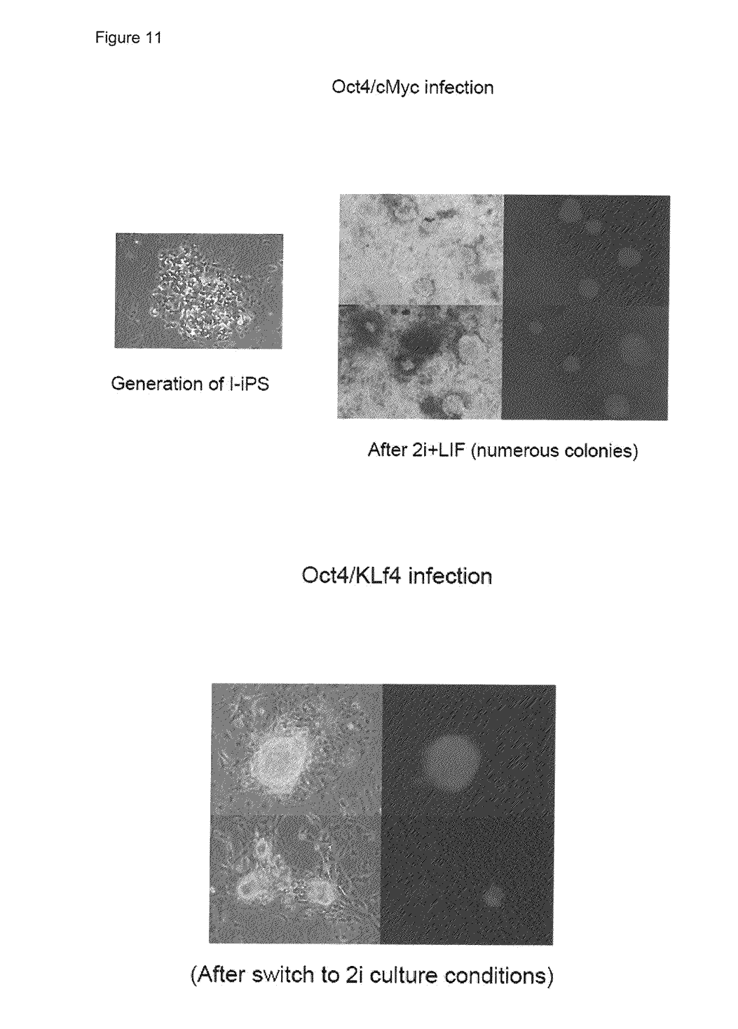

FIG. 11: shows reprogramming of cells using only 2 factors (Oct4 plus one other).

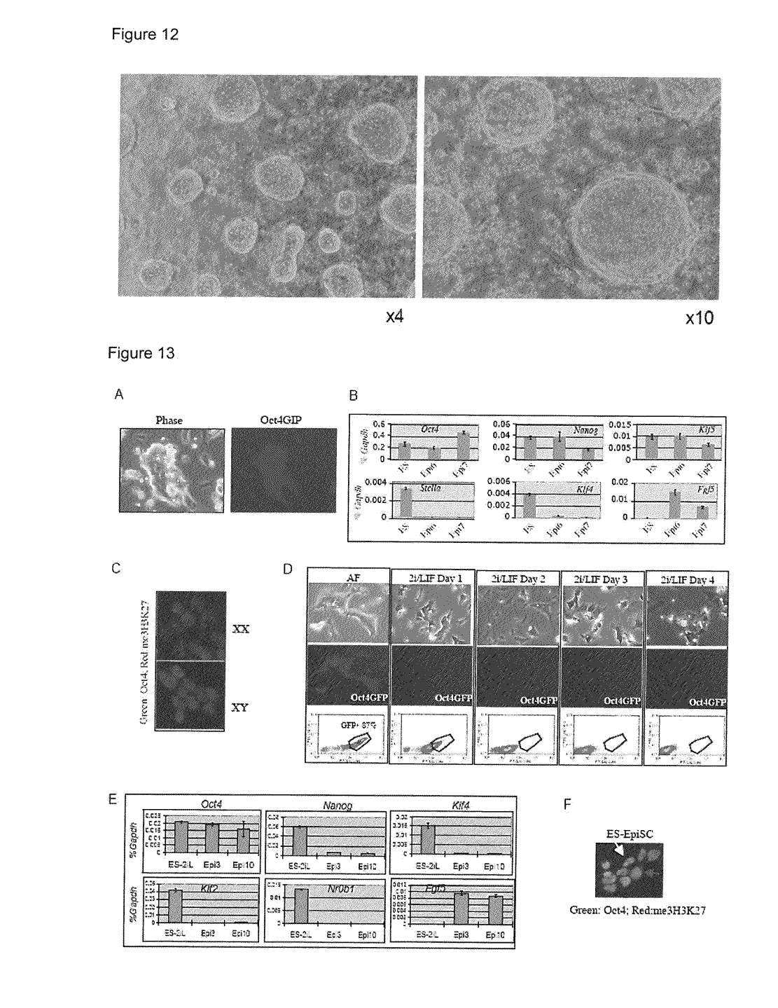

FIG. 12: stably transfected human induced pluripotent stem cells cultured in 2i plus LIF. Specifically the cells are shown are passage 8, after 2 months of continuous culture in 2i plus LIF.

FIG. 13: EpiSCs are distinct from and do not spontaneously convert to ES cells

A. Phase contrast and fluorescence images of established EpiSC cell line.
B. qRT-PCR analysis of marker gene expression in ES cells and EpiSCs. ES, ES cells in 2i/LIF. Epi6 and Epi7 are two independent EpiSC lines. Y-axis, relative expression to Gapdh.
C. Immunostaining of male and female EpiSCs for me3H3K27 and Oct4. White arrow indicates focus of staining diagnostic of an inactive X chromosome.
D. EpiSCs lose Oct4 expression and differentiate or die in 2i/LIF. AF, EpiSC cultured in activin A plus FGF2.
E. qRT-PCR analysis of ES cell differentiation into EpiSCs upon culture in bFgf and Activin. Epi3, Epi10 indicates cells in Fgf and Activin for three or ten passages respectively. Y-axis, relative expression to Gapdh.
F. Oct4 and me3H3K27 immunostaining of female ES cells derived EpiSCs. EpiSCs both express Oct4 and exhibit a nuclear body indicative of the inactive X.

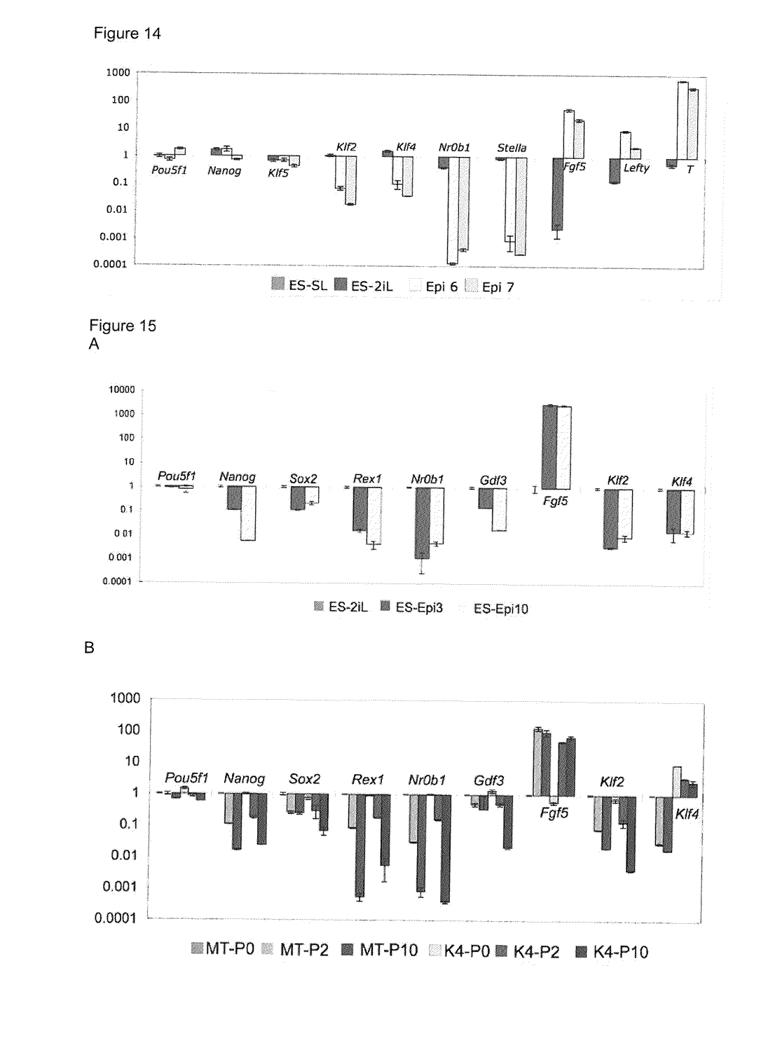

FIG. 14: qRT-PCR analysis of marker gene expression in embryo-derived EpiSCs compared to ES cells.

ES-SL, ES cells in Serum/LIP; ES-2iL, ES cells in 2i/LIF. Epi6 and Epi7 are two independent embryo-derived EpiSC lines. Y-axis, relative expression to Gapdh, then normalized to ES-SL

FIG. 15: qRT-PCR analysis of marker gene expression in ES cell derived EpiSCs (ES-Epi) compared to ES cells

A. qRT-PCR analysis of ES cell differentiation into EpiSCs upon culture in bFgf and Activin.
ES-Epi3, ES-EpilO indicates cells in Fgf and Activin for three or ten passages respectively.
Y axis is relative expression to ES cells in 2i/LIF (ES-2iL)
B. ES cells constitutively expressing Klf4 retain EpiSC marker profile in Fgf and Activin. MT, empty vector transfectants. PO, P2 and PIO, passage numbers in FGF/Activin. Y axis is relative expression to MT-PO, control vector transfected ES cells

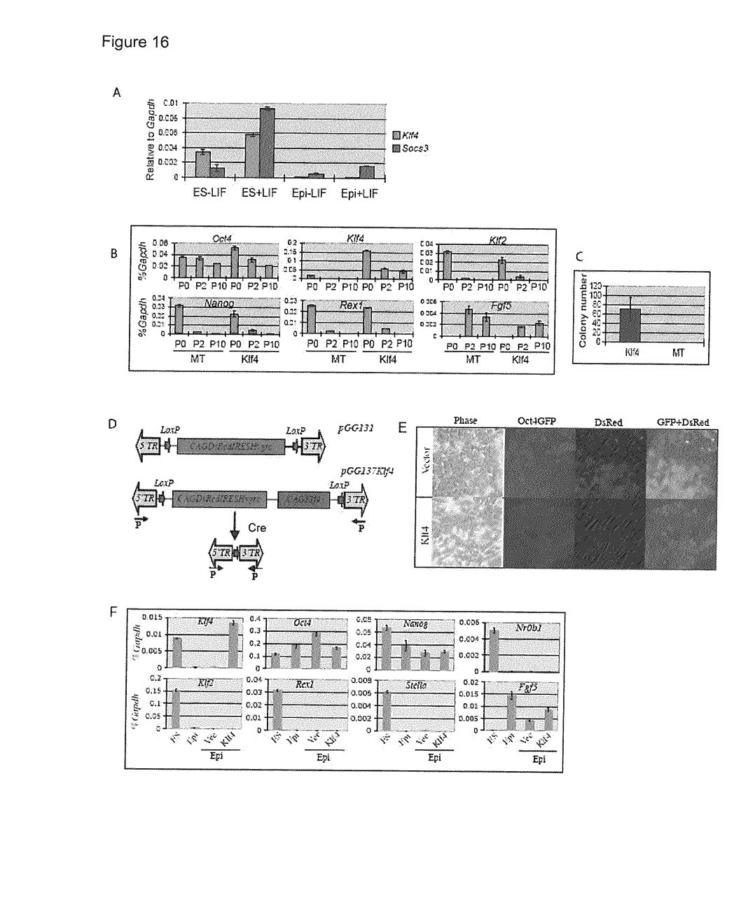

FIG. 16: Klf4 does not prevent ES cell differentiation into EpiSCs nor convert an EpiSCs population into ES cells in the presence of activin and FGF

A. qRT-PCR analysis of LIF induction of Klf4 in ES cells but not in EpiSCs. Cells were stimulated with LIF (+L) for one hour.
B. ES cells constitutively expressing Klf4 retain EpiSC marker profile in FGF2 plus activin
A. MT, empty vector transfectants. P0, P2 and P10, passage numbers in FGF/activin.
C Constitutive Klf4 expression permits continued recovery of ES cell colonies after culture activin and FGF.
D. PiggyBac vector for expression of Klf4 (pGG137Klf4) and control PiggyBac vector (pGG131). Arrows (P) indicates PCR primers amplifying the PB LTR fragment after Cre-mediated recombination.
E. Images of hygromycin selected Klf4 and control vector transfected EpiSCs.
F. qRT-PCR analysis showing forced Klf4 expression does not induce ES cell marker gene expression in EpiSC culture. ES, ES cells; Epi, EpiSCs; Vec, EpiSC transfected with control vector pGG131; Klf4, EpiSCs transfected with pGG137Klf4. Y-axis, relative expression to Gapdh

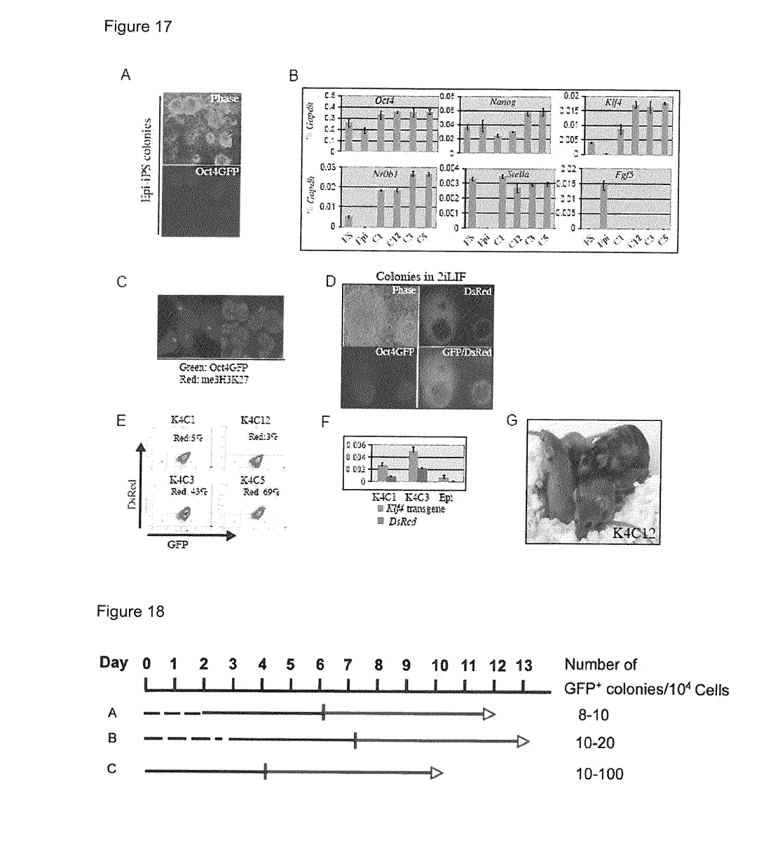

FIG. 17: EpiSCs transfected with Klf4 can convert to ground state pluripotency

A. Oct4 positive colonies generated by transfection with Klf4 and transfer to 2i/LIF after 72 hours. Images taken after 9 days in 2i/LIF.
B. qRT-PCR analysis of marker gene expression in ES cells, EpiSC cells and derivative Epi-iPS cells isolated in 2i/LIF. Y-axis, relative expression to Gapdh
C. me3H3K27 staining of female EpiSCs and derivative Epi-iPS cells.
D. Images of Epi-iPS colonies after 10 days in 2i/LIF, showing mutually exclusive expression of DsRed and OctGFP.
E. Flow cytometry analysis of four expanded Epi-iPS clones. Two clones retain weak but detectable red fluorescence.
F. qRT-PCR analysis of Klf4 transgene and Dsred expression in Epi-iPS cell clones and parental EpiSC cell line. Y-axis, relative expression to Gapdh.
G. Chimeric mice produced from the K4C12 Epi-iPS clone and agouti germline offspring

FIG. 18: time lines of transfection and Oct4-GFP+ve iPS cell colony formation after transfer to 2i/LIF.

A, cells plated in 2i/LIF 48 hours after transfection
B, cells plated in 2i/LIF 72 hours after transfection
C, Klf4 stable transfectants, transferred in 2i/LIF on day 0
Dashed line, time in Activin and Fgf
Black line, 2i/LIF
Blue line, 2i/LIF culture, GFP positive colonies expanding

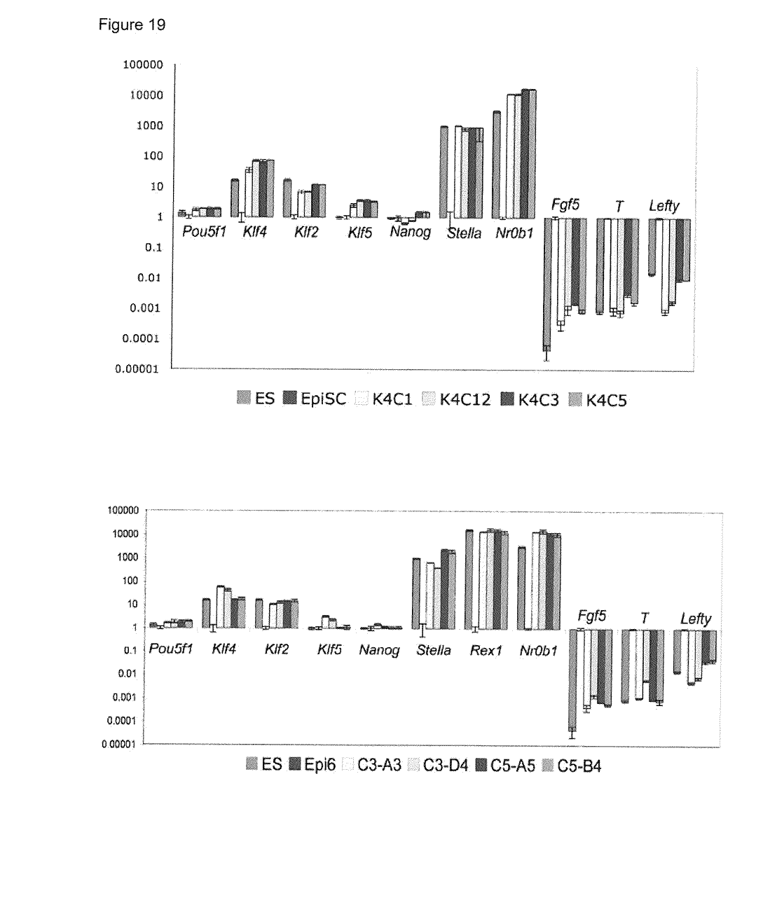

FIG. 19: qRT-PCR analysis of marker gene expression in Epi-iPS cells compared to embryo derived EpiSCs and ES cells

K4C1, K4C12, K4C3, K4C5 are clones with Klf4 transgene;
C3-A3, C3-D4, C5-A5, C5-B4 are clones with Klf4 deleted from K4C3 and K4C5.
Y-axis, relative expression to Gapdh, normalized to EpiSCs

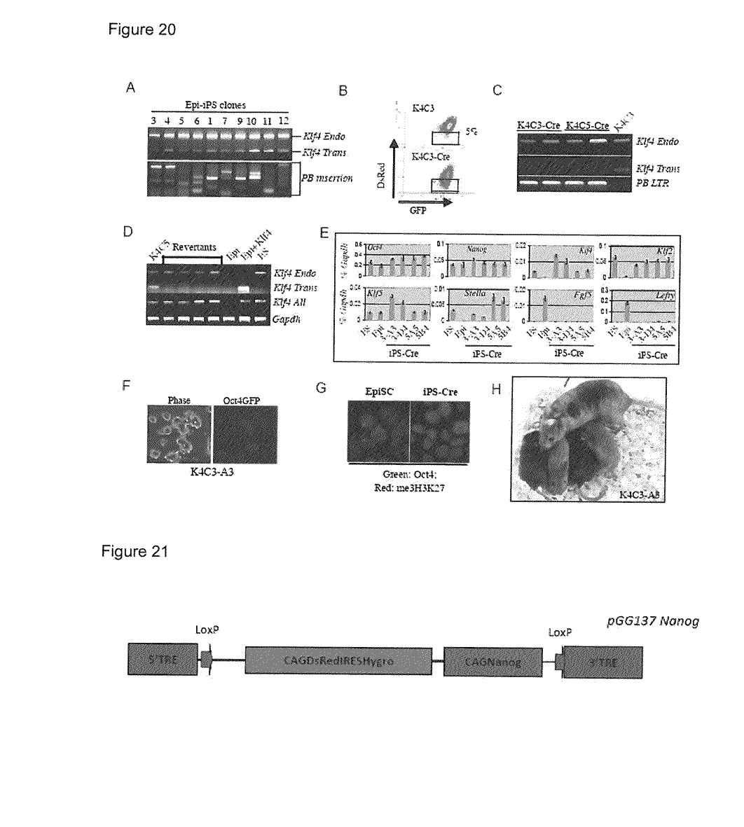

FIG. 20: Retention of ground state pluripotency after transgene excision

A. Splinkerette-PCR reveals 1 to 3 PB insertions in each iPS clone.
B. Flow cytometry showing DsRed negative population in K4C3 line before and after Cre transfection.
C. Genomic PCR showing loss of Klf4 transgene and gain of PB-LTR fragment in two revertant clones.
D. RT-PCR analysis showing lack of Klf4 transgene and Dsred expression in expanded Cre-reverted cells.
E. Marker gene expression in Cre-reverted cells from two parental Epi-iPS clones K4C3 and K4C5 compared to ES cells and EpiSC cells.
F. Images of a Cre-reverted Epi-iPS cell line.
G. m3H3K27 staining of Klf4 transgene deleted iPS cells compared with parental EpiSCs
H. Chimeric mice made with revertant K4C3-A3 cells and agouti offspring denoting germline transmission.

FIG. 21: piggyBac vector used for stable intergration of floxed Nanog transgene.

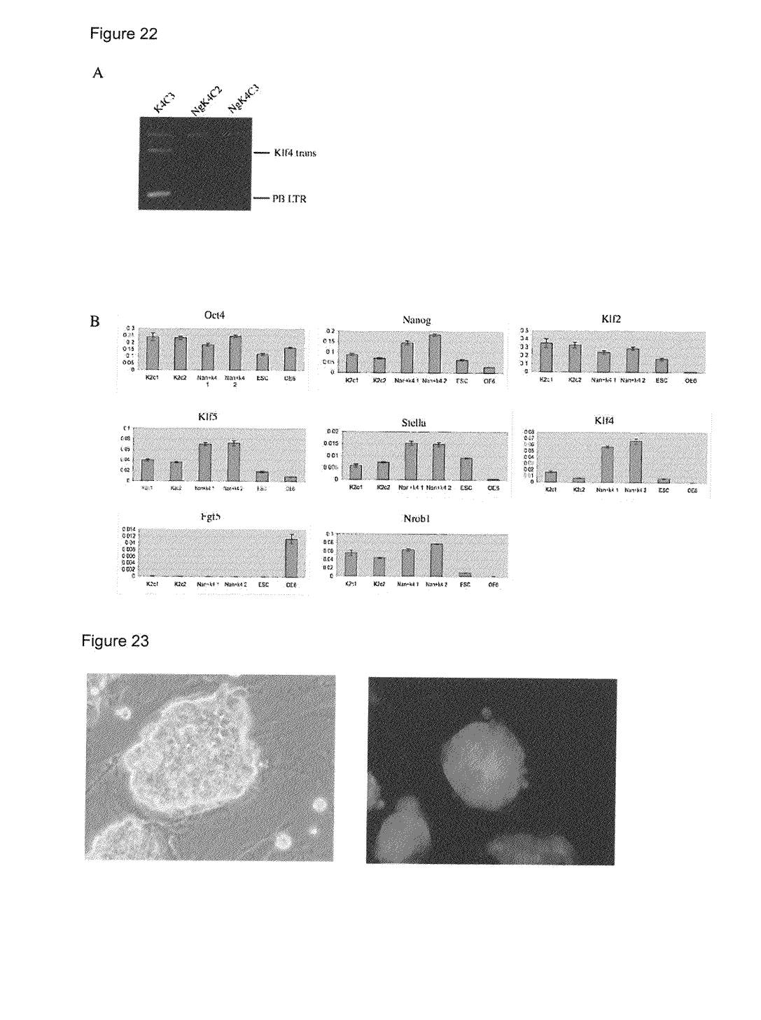

FIG. 22: results of transient transfection of mouse EpiSCs using PBNanog plus PBKlf4.

A. Gel image of PCR-amplified genomic DNA shows a Klf4 Epi-iPS cell clone (K4C3) sample containing the Klf4 transgene and PB LTR fragment, and two Epi-iPS cell clones (NgK4C2 and NgK4C3) generated by transient transfection with Nanog plus Klf4 that lack the Klf4 transgene and PB LTR fragment.
B. Histograms of qRT-PCR data for two Klf2 stably transfected Epi-iPS cell clones (K2C1 and K2C2) and two Nanog+K4 transient clones. Controls are ES cells and EpiSCs.

FIG. 23: image of human “ES” cells stably transfected with Nanog plus Klf4 that have been propagated in the presence of 2i and LIF without FGF or serum factors for over 1 month. The fluorescent image is the expression of dsRed linked to the Nanog transgene.

### EXAMPLE 1

### Material and Methods

### Mice

Target cells (NS cells and MEFs) were derived from HP165 mice, carrying regulatory sequences of the mouse Oct4 gene driving GFP and puromycin resistance.

### Cell Culture

NS cells were derived from 14.5-dpc foetal forebrain (F-NS) and adult lateral ventricle (A-NS) as described elsewhere (Conti et al., 2005). NS cells were maintained in N2B27 supplemented with 10 ng/ml of both EGF and FGF-2.

Foetal NS cells were also derived from a non-transgenic C57BU6 male foetus.

MEFs were isolated from 13.5 d.p.c. embryos. After the removal of the head, visceral tissues and gonads the remaining bodies were washed in fresh PBS, minced using a scalpel and then dissociated in a 0.1 mM trypsin/1 mM EDTA solution. Cells were collected by centrifugation (1200 rpm for 3 min) and resuspended in fresh medium. 1×10⁶ cells (passage 1) were cultured on T-25 flask at 37° C. with 5% CO₂. In this study, we used MEFs within three passages to avoid replicative senescence. MEFs were maintained in DMEM containing 10% FCS, 50 units ml⁻¹ penicillin, 50 μg ml⁻¹ streptomycin.

ES cells and iPS cells were cultured in GMEM containing 10% FCS, 1×NEAA, 1 mM sodium pyruvate, 5.5 mM 2-ME, 50 units ml⁻¹ penicillin and 50 μg ml⁻¹ streptomycin supplemented with leukemia inhibitory factor LIF (ES medium). As a source of LIF, we used conditioned medium (1:10,000 dilution) from COS cultures that had been transfected with a LIF-encoding vector.

N2B27 medium for serum free cultures was prepared as described (Ying and Smith, 2003). Inhibitors were used in combinations at the following concentrations: CHIR99021, 3 μM; PD0325901 1 μM or 2 μM. STO cells treated with mitomycin-C were used as feeder layer for the expansion of iPS cells.

Plat-E cells (Morita et al., 2000), which were used to produce retroviruses, were maintained in DMEM containing 10% FCS, 50 units ml⁻¹ penicillin, 50 μg ml¹ streptomycin, 1 μg ml⁻¹ puromycin and 10 μg ml⁻¹ blasticidin S.

### Retroviral Infection and iPS Cell Induction

Retroviral infection was performed as described previously (Takahashi and Yamanaka, 2006) with some modifications. Plat-E cells were seeded at 4×10⁶ cells per 100-mm dish. The following day, 9 μg of pMXs-based retroviral vectors for Oct3/4, Sox2, Klf4, or c-Myc were introduced separately into different Plat-E cells using 27 μl of FuGENE 6 transfection reagent. After 24 h, the medium was replaced with 10 ml of DMEM containing 10% FCS. Target cells (MEFs and NS cells) were seeded at 8×10⁵ cells per 100-mm dish or 1.2×10⁵ cells per 35-mm dish coated with gelatin or STO feeder layer. On the next day, virus-containing supernatants from these Plat-E cultures were filtered through a 0.45 μm cellulose acetate filter. Equal volumes of the supernatants were mixed and supplemented with polybrene at the final concentration of 4 μg ml⁻¹. NS cells were incubated in the virus/polybrene-containing supernatants for 24 h, after which the medium was changed with NS cell expansion medium supplemented with EGF and FGF-2 or DMEM containing 10% FCS, 50 units ml⁻¹ penicillin, 50 μg ml⁻¹ streptomycin. Three days after infection, the medium was changed with ES cell medium supplemented with LIF. For further expansion pre-iPS cells were replated onto feeders at day 5 in complete medium and passaged every few days by trypsinisation.

### Nucleofection

Transient transfection of pPyCAG plasmids was performed according to manufacturers' guidelines. Briefly, 2×10⁶ cells were harvested and collected by centrifugation (1200 rpm for 3 min). The pellet was resuspended at room temperature in Cell Line Nucleofector™ Solution V to a final concentration of 2×10⁶ cells/100 μl. The 100 μl cell suspension was mixed with 2-3 μg circular DNA (in 1-5 μl H2O or TE). The cells were transfected using the Nucleofector™ program U-20. The cells were transferred to prepared gelatin coated plates with DMEM containing 10% FCS, 50 units ml⁻¹ penicillin, 50 μg ml⁻¹ streptomycin. After 24 h the medium was changed to NS cell expansion medium supplemented with EGF and FGF-2. Three days after infection, the medium was changed to ES cell medium supplement with LIF.

### Plasmids

pMXs-gw plasmids; pMXs-Oct4, pMXs-Klf4, pMXs-cMycT58 and pMXs-Sox2 were obtained from Addgene repository. For transient transfections we used pPyCAG plasmids containing the following inserts: Klf4, Sox2, Oct4 and cMycT58.

### RT-PCR for Marker Genes

For RT-PCR, total RNA was extracted using RNeasy kit (Qiagen), and cDNA generated using superscript II (Invitrogen). PCR was performed using Taq Polymerase (Qiagen) or Phusion™ High-Fidelity DNA Polymerase (New England BioLabs). Primer sequences and cycling conditions are described in primer table (see below). Antibodies

For immunofluorescence (IF), the following antibodies and dilutions were used: Oct4 (1:100) from Santa Cruz Biotechnology (C-10, cat no: sc-5279), trimethyl H3-K27 (1:500) was a gift from Thomas Jenuwein, Nanog (1:200) from abcam (cat no: ab21603-100). Immunofluorescence was performed as previously described (Silva et al., 2003).

Analysis of genomic integration Genomic PCR for retroviral transgenes was carried out using the indicated primers. Southern hybridization was performed using the GE Healthcare AlkPhos Direct Labeling and Detection System with CDP-Star according to the manufacturer's specifications. Genomic DNA was digested with EcoRI and hybridized with full length Sox2 or Klf4 cDNA.

### Imaging and FACS

Slides were analyzed on a confocal microscope (Leica TCS SP5) and processed with Leica software and Adobe Photoshop. Images of live cells were captured with a Leica CTR microscope and processed with Leica software and Adobe Photoshop.

Flow cytometer analyses were performed using a Dako Cytomation CyAn ADP high-performance with FlowJo and summit software.

Primer table

| | | | Dsnat. | Ann. | Ext. | | |
|---|---|---|---|---|---|---|---|
| Gene | Forward | Reverse | Temp | Temp | Temp | Cycles | Maker |
| Nanog | CAGGTGTTTGAGG | CGGTTCATCATGG | 94 | 67 | 12 | 28 | Qiagen |
| | GTAGCTC | TACAGTC | | | | | |
| Endo Oct4 | TCTTTCCACCAGGC | TGCGGGCGGACA | 98 | 70 | 12 | 28 | Phusion |
| | CCCCGGCTC | TGGGGAGATCC | | | | | |
| Total Oct4 | GTGACTGCCTACCA | ATTGTCCGCATAG | 94 | 55 | 72 | 28 | Qiagen |
| | GAATGA | GTTGGAG | | | | | |
| FGF4 | CACGAGGGACAGT | CGTCGGTAAAGA | 94 | 58 | 72 | 28 | Qiagen |
| | CTTCTGG | AAGGCACA | | | | | |
| Rex1 (Zip42) | TTGGGGCGAGCTC | TTGCCACACTCTG | 94 | 57 | 72 | 28 | Qiagen |
| | ATTACTT | CACACAC | | | | | |
| Bibp | GGGTAAGACCCGA | ATCACCACTTTGC | 94 | 55 | 72 | 28 | Qiagen |
| | GTTCCTC | CACCTTC | | | | | |
| GAPDH | CCCACTAACATCAA | CCTTCCACAATGC | 94 | 55 | 72 | 28 | Qiagen |
| | ATGGGG | CAAAGTT | | | | | |
| Retro Oct4 | TGGTACGGGAAATC | AAGCTTAGCCAG | 98 | 62 | 72 | 35 | Phusion |
| | ACAAGTTT | GTTCGAGAAT | | | | | |
| Retro Sax2 | TGGTACGGGAAATC | AGTTGTGCATCTT | 98 | 62 | 72 | 35 | Phusion |
| | ACAAGTTT | GGGGTTCT | | | | | |
| Retro KI4 | TGGTACGGGAAATC | CGCTTCATGTGAG | 98 | 62 | 72 | 35 | Phusion |
| | ACAAGTTT | AGAGTTCCT | | | | | |
| Retro cMyc | TAAGGATCCCAGTG | TCTGCTGTTGCTG | 98 | 62 | 72 | 35 | Phusion |
| | TGGTGGTA | GTGATAGAA | | | | | |

### Results:

### Neural Stem Cells Undergo Efficient and Rapid Transformation Towards a Pluripotent State

We investigated whether neural stem (NS) cells derived from foetal (F) and adult (A) brain can be directly converted into ES like cells and if so, compare their conversion efficiency to mouse embryonic fibroblasts (MEFs), all using the known retrovirus-based methods. To do this we infected NS cells with the previously described minimal reprogramming factors Oct4, Sox2, cMycT58 and Klf4 (Takahashi and Yamanaka, 2006). At day 3 after infection a high proportion of cells exhibited already a cell morphology characteristic of ES cells.

Two days later plates were confluent and in need of passaging (FIG. 1a). Concomitantly with the acquisition of ES cell morphology gene expression analysis of both foetal F-NS and A-NS cells exhibited detectable expression of ES cell makers such as Fgf4, endogenous Oct4, Rex1 and Nanog (FIG. 1b). In contrast, expression of the same markers was not detected in infected MEFs at the time points analysed. The rapid conversion of NS cells towards a pluripotent state occurs even though infection efficiency and expression levels of the control transgene were shown to be similar to MEFs (FIG. 1c). A small proportion of infected NS cells exhibited also detectable Nanog protein (FIG. 1d). These results gave support to the notion that NS cells were permissive to nuclear reprogramming.

Using an antibody against the epigenetic marker trimethyl H3-K27 (me3H3K27) (Silva et al., 2003) we analysed the X chromosome status in female NS cells at day 5 after infection. The lack of the silent X chromosome in female cells is an exclusive epigenetic feature of pluripotent cells. However, a clear nuclear body was detected in every single nucleus, showing that reactivation of the silent X chromosome had not occurred (FIG. 1e). Therefore, despite acquiring some similarities to ES cells, these iPS cells have not reprogrammed fully at day 5.

### Investigation of Partially Reprogrammed State

To investigate whether further reprogramming might occur in a small proportion of cells we examined expression of an Oct4-GFP transgene. This has been shown to reactivate when the differentiated genome acquires pluripotency (Silva et al., 2006; Ying et al., 2002). In MEFs, Oct4 reporter activity is not detectable until 3 weeks after infection (Meissner et al., 2007). For NS cells, approximately 2% of cells expressed detectable GFP only 5 days after infection. We purified GFP positive cells by flow cytometry and expanded them on a feeder layer. In this population both Fgf4 and endogenous Oct4 mRNA are detectable at similar levels as in ES cells. Fbx15 and NrOb1 transcripts are also present, although at lower levels. However, Nanog protein remains absent and the inactive X chromosome persists (results not shown). Total Oct4 mRNA levels were also elevated compared to ES cells, indicating continued activity of the retroviral transgene. Moreover, Oct4 reporter expression was unstable and lost within 2 passages. A population expressing GFP could be maintained by repeated FACS isolation but blastocyst injection did not yield chimaeras, confirming that full pluripotent status is not attained. These observations indicate that neither morphology nor Oct4 reporter activation are reliable surrogates per se for designation of pluripotent identity. Indeed we found that infections of MEFs yielded colonies of ES cell-like morphology that could readily be expanded but remained negative for expression of endogenous pluripotency genes and did not stably activate the Oct4-GFP reporter. Interestingly, these colonies emerged at around 14 d, while rarer colonies that did express Oct4-GFP only appeared from 3 wk onwards. These later colonies appear to arise independently from the earlier emerging pre-iPS cell colonies. We surmise that this represents a delayed, low-efficiency, “stochastic” path to iPS cell generation.

Interestingly we also noted a strong effect of genetic background on reprogramming frequency. MEFs from inbred strain 129 infected with four factors yielded many more ES cell-like colonies and subsequently 20-fold more 2i-iPS cell colonies (not shown) than the Oct4-GFP MEFs, which are on a mixed, predominantly outbred MF1 background. It is of interest that 129 is the mouse strain from which ES cells are most readily obtained by blastocyst culture

NS Cells are Converted by the Reprogramming Factors into a Heterogeneous Cell Population with Properties of EpiSCs

To further investigate the phenotype of these partially reprogrammed cells we assessed Oct4 reporter activity in different culture environments. In conventional ES cell culture using the self renewal cytokine Leukaemia Inhibitory Factor (LIF) and serum in presence or absence of feeders, Oct4-GFP expression was not maintained. In contrast, reporter expression stabilized when cells were cultured serum free in the presence of Fgf2 and activin (FIG. 2). These are conditions used for the propagation of EpiSCs derived from post-implantation embryos (Brons et al., 2007; Tesar et al., 2007). Dependency on signaling through the activin receptor was confirmed by loss of reporter activity, cell death and differentiation in presence of the inhibitor, SB43154212. We conclude that NS cells are efficiently converted by the reprogramming factors into a heterogeneous cell population with certain properties of EpiSCs rather than ES cells.

MEFs also yielded similar incompletely reprogrammed products.

This shows that purifying the Oct4-GFP positive sub-population was not sufficient to yield authentic pluripotent stem cells under the conditions applied.

A rationale is that Oct4 expression is not exclusive to the ES cell stage and is also present in EpiSCs in culture and throughout the egg cylinder in the post-implantation embryo.

### NS Cells are Responsive to 2i 3 Days After Infection

A stringent selection to overcome the appearance of undesirable cell types is through the use of Nanog expression as selection (Maherali et al., 2007; Okita et al., 2007; Wernig et al., 2007).

However, we tried a different strategy which used defined culture conditions for the derivation and expansion of pluripotent cells (Ying et al., Submitted). This uses serum-free medium containing small molecule inhibitors of the MEK/Erk (extracellular signal regulated kinases 1 and 2) pathway and glycogen synthase kinase-3 (medium containing a MEK inhibitor and a GSK3 inhibitor is referred to as 2i or 2i medium). We further included LIF. In addition, because of the reliance on Fgf/Erk activities for cell survival and proliferation, other cell types, including most somatic cell types and EpiStem cells, die in 2i culture conditions. Therefore, we asked whether secondary cell types can be eliminated and bonafide pluripotent cells isolated using 2i.

Infection of both A-NS cells and MEFs was carried out as previously, but media was replaced with 2i at days 3 or 5 after infection. Non-infected control cells died or stop proliferating with the addition of 2i. In contrast, numerous infected NS cells responded to 2i culture conditions and emerging colonies were observed in wells where 2i was applied 3 days after infection (FIG. 3a). The number of colonies increased when 2i was applied at day 5.

Similar results were obtained from foetal NS cells including from a wild-type strain C57BL/6-derived line without recourse to any transgene reporter.

MEFs showed almost no response, only the odd colony in wells where 2i was applied 5 days after infection and more than 100 fold greater than that obtained from MEFs. Cell colonies stained positively for alkaline phosphatase and in a proportion of these reactivation of the Oct4-GFP reporter transgene occurred (FIG. 3b).

The latter could be expanded without loss of Oct4 reporter activity and cell lines with ES cell morphology established (2i-iPS) (FIG. 3c). These were also obtained from F-NS cells and from MEFs. Characterization of 2i-iPS cells revealed that Nanog and Rex1 are expressed and levels comparable to wt ES cells (FIG. 4a). Immunofluorescence analysis showed the expected nuclear pattern for Nanog protein (FIG. 4b).

Epigenetically, 2i-iPS cells showed also reactivation of the silent X chromosome as seen by the absence of me3H3K27 nuclear body (FIG. 4c) and by expression of Tsix from both X chromosomes (not shown). When 2i-iPS cells were cultured in serum culture conditions without the leukaemia inhibitor factor (LIF) GFP expression was lost and cells differentiated, mimicking wt ES cell differentiation (FIG. 5a).

Undifferentiated 2i-iPS cells could be maintained by Leukaemia Inhibitory Factor (LIF) and BMP, however, mimicking ES cell responsiveness, and contrasting with EpiScs (Brons et al., 2007; Tesar et al., 2007) and to Fbx15 iPS cells2 which are unresponsive to LIF.

To ultimately test the potency of 2i-iPS cells we checked the ability of these cells to contribute to the animal. As a result of blastocyst injection we obtained chimeric animals (FIG. 5b). This establishes that the reprogrammed cells can respond fully to instructive cues in the developing embryo and have authentic pluripotency.

In summary, the existing retrovirus-based methods did not yield truly pluripotent cells but cells which were only partially reprogrammed (as shown in e.g. by a silent X chromosome). These partially reprogrammed cells were fully reprogrammed by 2i. NS cells responded more rapidly to 2i culture conditions than MEFs. Importantly, 2i allowed the conversion of known iPS cells into 2i iPS cells with gene expression and epigenetic similarities to ES cells and eliminated secondary cell phenotypes, the need for feeders and gene selection. Significantly, 2i A-NS-iPS cells showed in vitro and in vivo pluripotency.

### 2i Promotes Nuclear Reprogramming

The isolation of 2i-iPS cells raised the question whether the culture conditions act stochastically by selecting cells where full reprogramming has occurred or by promoting nuclear reprogramming.

To address this, we expanded individual colonies from infection of both MEFs and NS cells by conventional culture on a feeder layer in serum and LIF. As described above, these cells exhibit uniform ES cell-like morphology but do not express the Oct4-GFP reporter, Nanog, or Rex1. After five or more passages, cells were plated at clonal density and cultured for 7 d until macroscopic colonies were evident. Colonies were counted and duplicate wells either replenished with serum-containing medium or transferred to 2i/LIF medium. After a further 7 d activation of the Oct4 reporter, transgene was observed only in 2i/LIF (FIG. 6a). All colonies from the MEF-derived clone became Oct4-GFP-positive, and 81% (129/159) from the NS cell derived clone became Oct4-GFP-positive. These populations were passaged and expanded. They express Nanog (FIG. 5b) and Rex1 (not shown), both of which remained absent in the cells maintained in serum. The speed and high incidence of conversion without major cell death indicates an inductive process. To confirm this, we documented transition of a typical colony from Oct4-GFP-negative to -positive status over a period of 4 d in 2i culture. It is noteworthy that the progressive gain of Oct4-GFP throughout the continuously expanding colony is a reciprocal profile to the dynamic loss of Oct4 observed in undifferentiated inner cell masses explanted in culture without Mek inhibition. In both cases, change in Oct4 is not linked to any overt morphological changes.

We also examined the effect of adding inhibitors in the presence of foetal calf serum plus LIF. The addition of 2i induced stable activation of Oct4-GFP in around 10% of emerging colonies. GSK3 inhibitor alone had no discernible effect, but the MEK inhibitor was comparable to 2i. The reduced frequency compared with serum-free 2i culture may be because the ability of PD0325901 to suppress Erk activation is counteracted by serum factors. Nonetheless, cells expanded in serum and LIF with either MEK inhibitor only or both inhibitors acquired expression of Nanog and Rex1.

These findings indicate that factor-induced reprogramming primarily generates an undifferentiated but nonpluripotent cell state. This is a pre-pluripotent (pre-iPS) condition, however, and the final transition is efficiently induced by blockade of Erk signaling in conjunction with stimulation by LIF. GSK3 inhibition consolidates this process. We surmise that the 2i/LIF regime completes the reprogramming process by imposing a ground state upon pre-iPS cells.

Specifically, it appears that 2i acts by promoting final stage nuclear reprogramming i.e. the speed and high incidence of conversion indicates that this is an inductive rather than a selective phenomenon.

### Exogenous Sox2 is not Required for 2i Mediated Conversion of NS Cells

Induction of the reprogramming cascade has been demonstrated to require two core genes, Oct4 and Sox2, in combination with either Klf4 and optionally cMyc (Okita et al., 2007; Takahashi et al., 2006) or with Lin28 plus Nanog (Yu et al., 2007). Interestingly, analysis of 2i-iPS clones for retroviral integration revealed that Sox2 was absent in some cases (FIGS. 10a and 10b). These cells yielded chimeric mice (FIG. 10c), indicating that authentic 2i-iPS cells may be obtained without exogenous expression of Sox2.

In order to confirm this, we infected adult NS cells with Oct4, Klf4 and cMycT58 without Sox2. As with four factors, three factor infected NS cells rapidly and efficiently convert into I-iPS cells that do not stably activate endogenous Oct4 or Nanog when maintained in serum on feeders.

However, unlike four factor infected I-iPS cells which exhibit considerable differentiation during expansion, three factor infected cells maintain more homogenous ES cell-like morphology (data not shown). This suggests that exogenous expression of Sox2 destabilises I-iPS cells and induces differentiation. Importantly, when transferred to 2i, three factor infected I-iPS produced numerous colonies most of which activated the Oct4 reporter transgene (FIGS. 10e and i). These were subsequently expanded and lines established (FIG. 10f). Their profile is similar to ES cells and other 2i-iPS cells in expression of Rex1 and Nanog and absence of an inactive X chromosome (FIG. 10h).

Very high efficiency of reprogramming was obtained using only 3 factors and direct application of the 2i-medium. Chimera-forming iPS efficiency was approximately 1 in 200 for triple infected cells (1200 colonies/0.7×0.7×0.7×8×105 (plated cells))

Thus, exogenous Sox2 is not required for 2i mediated conversion of NS cells. This may be because NS cells already express Sox2 (FIG. 10d). Consistent with other reports we have not obtained iPS cells from MEFs without Sox2 infection. Importantly, however, our data indicate that endogenous levels of Sox2 in NS cells, which are similar or slightly lower to those found in ES cells [10], are sufficient. Moreover, elevated expression of Sox2 may destabilise pre-iPS cells and favour their differentiation.

Nonetheless, Oct4 is left as the only exogenous factor required in all examples of iPS cell generation. As shown below, two factors may be advantageously used e.g. Oct 4 plus cMyc or Klf4, where these 2^{nd} factors may act as facilitators of the reprogramming.

These findings establish that NS cells can be triggered to undergo rapid conversion to full induced pluripotency at a frequency orders of magnitude higher than for fibroblasts. Furthermore NS cells can be fully converted without the need for exogenous Sox2 expression. Therefore the somatic cell context determines different efficiencies and requirements for nuclear reprogramming.

Although the preexistence of Sox2 in NS cells may be an important factor, the results herein nevertheless demonstrate that tissue stem cells may generally be a favoured substrate for reprogramming.

### Oct4 and Klf4 are Sufficient for NS Cell Conversion to Pluripotency

Previous studies have shown that fibroblasts can be reprogrammed using Oct4, Klf4, and Sox2, without necessity for c-Myc (Takahashi K, Yamanaka S (2006); Nakagawa et al. 2008). We therefore tested the combination of just two factors, Oct4 and Klf4. Without c-Myc, the rapid transition to undifferentiated morphology was not observed. However, ES cell-like colonies did appear between 2 and 3 wk post-infection. Approximately 100 colonies emerged per 8×10⁵ plated cells. These were more heterogenous in morphology than three factor-infected cells. Nonetheless, upon transfer into 2i, around one-third of the colonies stably activated Oct4-GFP and acquired the features of 2i-iPS cells, including the cardinal attribute of contributing to adult chimaeras (not shown).

NS Cells can be Converted into ES-Like Cells without Genomic Incorporation of Reprogramming Factors

The observation that the dynamics of NS cell conversion towards a pluripotent state were rapid and that 2i promoted reprogramming led us to investigate whether reprogramming could be achieved without the need for genomic incorporation of the four factors. To attempt this we transfected circular plasmids containing the four reprogramming factors (Oct4, Sox2, Klf4, C-Myc; “4F”) into adult mouse derived NS cells (O4G) using the Amaxa Nucleofector. The transfected cells were cultured for two days in NS cell culture medium supplemented with FGF2 and EGF. At day three we switched to ES culture medium containing serum and LIF. After 5 days we analysed the cell population for Oct4 reporter activity but this was GFP negative (data not shown).

However, because the Oct4 reporter transgene might take longer than 5 days to reactivate we replated transfected cells on a feeder layer for later analysis. Surprisingly, colonies emerged but these were negative for GFP expression. Morphologically cells resembled GFP negative pre-iPS clones. After expansion in ES cell medium colonies exhibiting an ES cell-like morphology were picked and separately expanded. At day 21 we replaced the ES culture medium with serum-free 2i and LIF or serum-replacement (KSR) 2i and LIF medium. and LIF. Interestingly, cells did not die and reactivation of the Oct4 reporter was detected in both conditions around 5-8 days after the final medium switch. Clonal lines were derived using single cell deposition after GFP cell sort. These cells expanded and resembled ES cells cultured in 2i and LIF (FIG. 7a). To confirm that the cells did not incorporate the plasmids we performed genomic PCR. This has shown that for the 3 plasmids analysed, cMyc, Klf4 and Oct4, no genomic incorporation occurred (FIG. 7b). Furthermore RT-PCR confirmed expression of ES genes at similar levels to ES cells (not shown). Southern blot analysis showed no incorporation of any of the four transfected plasmids in one out of two derived iPS clonees. This is shown in FIG. 7c wherein iPS cells were obtained by transient expression of the four reprogramming factors and subsequent culture in 2i+LIF. Clonal lines 1-3 from colony O4G-4F1 show no genomic integration of the four transfected plasmids (left). However, clonal lines 1-3 of colony O4G-4F2 show genomic incorporation of Oct4, Klf4 and C-Myc, but not Sox2 (right, red arrows).

In conclusion, NS cells underwent rapid and efficient conversion towards a pluripotent state. Importantly, known iPS cells were shown not to be fully reprogrammed, and 2i then promoted nuclear reprogramming and eliminated: (i) secondary cell phenotypes, (ii) need for feeders, (iii) need for gene selection. In addition, NS cells were converted to a pluripotent state without genomic incorporation of reprogramming factors.

The invention thus provides methods for reprogramming cells to yield reprogrammed, pluripotent cells without genetic modification and also truly pluripotent cells which can be maintained in culture for many passages without loss of pluripotency.

### EXAMPLE 2

We modified the methods used for reprogramming of mouse NS cells for reprogramming of human adult NS cells (XX) using transient plasmid-based expression of the reprogramming factors of example 1.

Plasmid preparation

| | | | |
|---|---|---|---|
| Plasmid | Amount | Volume | 10 rx |

| | | | | | | |
|---|---|---|---|---|---|---|
| CAG Oct4 | 0.2 | μg | 0.09 | μl | 0.9 | μl |
| CAG cMyc | 1 | μg | 0.26 | μl | 2.6 | μl |
| CAG Klf4 | 1 | μg | 1 | μl | 10 | μl |
| CAG Sox2 | 1 | μg | 0.52 | μl | 5.2 | μl |
| CAG Nanog | 1 | μg | 0.52 | μl | 5.2 | μl |

| | | | | | |
|---|---|---|---|---|---|
| CAG GFP | 1 | μg | 1 | μl | — |

### Nucleofection Protocol

hNS cells were expanded on laminin using SCS RHBA media supplemented with EGF and FGF.

On the day of nucleofection:—
Cells were dissociated into a single cell suspension using Accutase.
8×10⁸ cells were separated and spun at 1400 rpm for 3 min.
Cells were resuspended in 400 μl of Amaxa Nucleofection Solution V.
2.39 μl of mixed DNA (or 1 μl of CAG GFP plasmid) was added to the Nucleofection cuvette as well as 100 ul of the cells suspension (2×10⁸ cells).
Cuvette was placed in Nucleofector and run at T-20 program.
Nucleofected cells were transferred from the cuvette into prepared and equilibrated gelatin coated 10 cm plates containing DMEM+10% FCS using the supplied pipette.
Two nucleofection reactions were placed into same 10 cm plate (Total of 4×10⁶ cells per plate).

### Day 1

Media was changed to N2B27 supplemented with EGF and FGF

### Day 3

Media was changed to GMEM+FCS+Lif

### Week 2-Week 3

Colonies emerged

### Week 4

Colonies were picked onto Mitomycin C inactivated STO feeders (48 well plate) [111207] in GMEM+FCS+Lif

### Week 7

Media was changed to N2B27 supplemented with 1 μM PD03+Chiron+Lif (2i medium)

Colonies were obtained with ES cell morphology (FIGS. 8, 9). Hence, reprogrammed human cells were obtained, not genetically modified compared with the starting NS cells. These cells were maintained for repeated passaging in 2i-containing media. Sample colonies are shown in FIGS. 8 and 9; the colonies of human cells are in the middle surrounded by the debris of cells which failed to reprogram and which died in the 2i media.

### EXAMPLE 3

In further experiments, infection of NS cells with only 2 factors (Oct4 and cMyc) resulted in the generation of incompletely reprogrammed iPS cells (I-iPS) as with 3 or 4 factors (above). Nevertheless once these were cultured in 2i+LIF conditions numerous fully pluripotent iPS cell colonies appear with high efficiency (as demonstrated by GFP reporter expression—see above).

In other 2-factor experiments, Oct4 and Klf4 I-iPS colony appearance was delayed (2 weeks for colony appearance). However, when 2i+LIF was applied directly (without culture on feeder) a number of GFP colonies appeared.

The results are shown in FIG. 11.

### EXAMPLE 4

As shown in the Examples above, NS cells can be converted to a partially reprogrammed state using fewer than 4 factors.

In the next Example, the inventors further demonstrated the generation of human induced pluripotent stem cells (having authentic “ES” properties) by piggyBac mediated stable transfection and culture in 2i.

### Materials

D-MEM/F-12 (invitrogen 21331)
200 mM L-GULUTAMINE 100× (invitrogen 25030)
10 mM MEM Non Essential Amino Acids 100× (invitrogen 11140)

### 2-MERCAPTOETHANOL (Sigma M3148)

### Recombinant Human FGF-basic (PEPROTECH 100-18B)

KNOCKOUT Serum Replacement (invitrogen 10828)

### Dulbecco's PBS(1×) (PAA H15-002)

### RHB-A (Stem Cell Science SCS-SF-NB-01)

### Recombinant Murine EGF (PEPROTECH 315-09)

Accutase solution (Sigma A6964)

### Laminin (Sigma L2020)

NEUROBASAL medium (invitrogen 21103)
B27 supplement (invitrogen 17504)

### N2

LIF recombinant human (MILLIPORE LIF1005)
Hygromycin B (invitrogen 10687)

### Human Neural Stem Cell Medium

### RHB-A

10 ng/ml basic FGF
10 ng/ml EGF

### Human iPS Cell Induction Medium

### D-MEM/F-12

### 20% KNOCKOUT Serum Replacement

### 2 mM L-GULUTAMINE

### 0.1 mM MEM NEAA

### 0.1 mM 2-MERCAPTOETHANOL

10 ng/ml basic FGF

### Human Ground State Medium (2i Plus Human LIF Medium)

### 500 ml D-MEM/F-12

500 ml NEUROBASAL medium
10 ml B27 supplement

### 5 ml N2

### 2 mM L-glutamine

### 0.1 mM 2-MERCAPTOETHANOL

10 ng/ml LIF recombinant human

### 3 μM CHIR99021

### 1 μM PD0326901

The open reading frames of human Oct4 and Klf4 genes were sub-cloned into pDONR221 (invitrogen). Both cDNAs were then transferred using the Gateway cloning system (invitrogen) to a piggybac (PB) vector containing a CAG promoter and hygromycin selection marker.

Human Neural stem (NS) cells (cell lines; CB660 and CB541) were maintained in human neural stem cell medium on laminin-coated dishes. After they became confluent, they were rinsed in PBS and dissociated using accutase. 2 μg of PBase, 2 μg of PB containing human Oct4 and 2 μg of PB containing human Klf4 were transfected into 2×10⁶ hNS cells using the Amaxa nucleofection system. Nucleofection was performed essentially according to the manufactured protocol, using Cell Line Nucleofector Kit V (VCA-1003) and program T-020. After nucleofection, cells were seeded on a laminin-coated 10 cm dish and cultured in hNS medium. One day after nucleofection, 200 μg/ml hygromycin B was added in hNS medium to select for stable transfectants.

After 12 days of selection, 7×10³ surviving cells were transferred to a new 10 cm dish on MEF feeder cells and were then cultured in human ES cell medium. Two weeks later colonies began to emerge and human ES cell medium was changed to 2i plus LIF medium. After 10 days in 2i plus LIF medium, rounded ES cell-like colonies were picked and passaged onto fresh MEF feeder dishes. These cells were able to be expanded in 2i plus LIF medium for more than 6 passages over 2 months of continuous culture. The colony morphology remains similar to mouse ES cells with no overt differentiation (FIG. 12).

### Figures

### EXAMPLE 5

In the next Example, the inventors demonstrate regeneration of a naïve ground state pluripotency from mouse Epistem cells (EpiSCs) using a single transgene which can be deleted after the reversion.

### Background

In the mouse pluripotent stem cell lines can be established from two distinct phases of early development (Rossant, 2008). Embryonic stem (ES) cells are obtained from naive epiblast in pre-implantation blastocysts (Batlle-Morera, 2008; Brook and Gardner, 1997; Evans and Kaufman, 1981; Martin, 1981). Epistem cells (EpiSCs) are derived from columnar epithelial epiblast of the early post-implantation embryo (Brons et al., 2007; Tesar et al., 2007). ES cells retain character of early epiblast and can be incorporated into the host embryo when injected into blastocysts (Gardner and Rossant, 1979). They subsequently contribute to all lineages of the developing and adult mouse (Beddington and Robertson, 1989; Bradley et al., 1984; Gardner and Rossant, 1979; Nagy et al., 1993). In contrast neither freshly isolated post-implantation epiblast cells nor EpiSCs are capable of functional colonisation of a host blastocyst (Rossant et al., 1978; Tesar et al., 2007).

Both ES cells and EpiSCs are capable of multilineage differentiation in vitro and can form teratomas when grafted into adult mice (Brons et al., 2007; Tesar et al., 2007). Both cell types express the three transcriptional regulators, Oct4, Sox2 and Nanog, generally considered to constitute the core pluripotency network (Boyer et al., 2005; Loh et al., 2006; Wang et al., 2006). However, there are significant differences in gene expression between ES cells and EpiSCs (Tesar et al., 2007).

The molecular basis for restriction of egg cylinder epiblast and EpiSCs compared with naïve epiblast and ES cells is presently unclear.

Furthermore, the culture conditions for maintaining the two cell types are quite distinct:

ES cells self-renew in response to the cytokine leukaemia inhibitory factor (LIF) (Smith et al., 1988; Williams et al., 1988) and either serum, bone morphogenetic protein (Ying et al., 2003), or inhibition of Mek/Erk signalling (Burdon et al., 1999; Ying et al., 2008). They are driven into differentiation by FGF/Erk signalling (Kunath et al., 2007; Stpyridis et al., 2007).

EpiSCs in contrast are maintained by FGF and activin (Brons et al., 2007).

### Material and Methods

### EpiSC Derivation and Culture

EpiSCs were derived from homozygous HP165 embryos carrying the Oct4GiP (eGFPiresPuro) transgene (Ying et al., 2002) maintained on a hybrid 129xMF1 strain background, and from inbred strain 129 embryos.

EpiSCs were derived from E5.75 embryos using activin A (20 ng/ml) and FGF2 (12 ng/ml) essentially as described (Brons et al., 2007a) except that we employed N2B27 medium (Ying and Smith, 2003). OE cell lines were derived from embryos carrying an Oct4GiP (eGFPiresPuro) transgene (Ying et al., 2002). EpiSCs were also derived from non-transgenic strain 129 embryos. Cells were used between 10 and 25 passages.

Differentiated cells could be eliminated as required from OE cultures by puromycin (1 μg/ml) selection for expression of the Oct4GiP transgene.

### Embryonic Stem Cell and Induced Pluripotent Stem Cell Culture

2i/LIF comprises the Mek inhibitor PD0325901 (1 μM), the GSK3 inhibitor CHIR99021 (3 μM), and leukaemia inhibitory factor (LIF, 100 U/ml) in N2B27 medium (Ying et al., 2008). Cells cultured were expanded by dissociation with trypsin and replating every three days.

ES cells overexpressing Klf4 were generated by electroporation of Oct4βgeo reporter cells (IOUD2) (Burdon et al., 1999) with a pPyCAGKlf4iP construct followed by puromycin selection (1 μg/ml). For ES cell to EpiSC differentiation, cells were plated at a density of 1-2×10⁴ per cm² in fibronectin coated plates. 24 hours after plating medium was changed to N2B27 with activin A and FGF2. Thereafter cells were maintained in EpiSC culture conditions and passaged every 2-3 days. For colony formation, 1000 cells were plated per well in fibronectin-coated 6-well plates in activin/FGF2 or in laminin coated plates in 2i/LIF. After 6 days, colonies were fixed and stained for alkaline phosphatase. Colonies were scored using Image J software.

### PiggyBac Vector Transfection

A piggyBac backbone vector (pGG84) containing two loxP sites was prepared by PCR from PB-SB-Neo (Wang et al., 2008). The CAG promoter and DsRedMSTiresHygro cassette were amplified by PCR and inserted into pGG84 by three-way ligation to generate the control PB vector (pGG131). A CAG-DEST-bGHpA cassette was inserted into pGG131 by restriction enzyme digestion and ligation to generate a Gateway destination gene expression vector pGG137. The relative gene-coding region was amplified by PCR from mouse embryonic stem cell cDNA, sequencing confirmed and Gateway cloned into pGG137 to generate the final PB expression vector.

To establish PB transgenic EpiSC lines, 1×10⁶ cells were co-transfected using Lipofectamine™ 2000 (Invitrogen) with 1 μg of pGG137Klf4 or control pGG131 vector plus 2-3 μg PBase expressing vector, pCAGPBase (Wang et al., 2008). Transfection efficiency was evaluated by flow cytometry for dsRed expression. To select for stable transfectants hygromycin (200 μg/ml) was applied for at least 5 days. To delete transgenes, 1×10⁵ cells were transfected with 1 μg of Cre expression plasmid using Lipofectamine™ 2000. Five days after transfection dsRed negative cells were purified and individually deposited into a 96 well plate using a MoFlo® high-performance cell sorter (DakoCytomation). After expansion genomic PCR was employed to identify revertants lacking the Klf4 transgene. RT-PCR was used to confirm the lack of Klf4 transgene and DsRed expression.

### iPS Cell Induction and Propagation

EpiSCs, either stable transfectants isolated after hygromycin selection, or cells immediately after transfection, were plated at a density of 1×10⁴, 5×10⁴, and 1×10⁵ per well of 6-well tissue culture plates in EpiSC culture condition. After 24 hours, medium was replaced with 2i/LIF and subsequently refreshed every other day. The number of Oct4GFP positive clones was manually counted using fluorescence microscopy. ES cell-like clones were picked after 14 days in 2i/LIF and subsequently expanded by accutase dissociation and replating every three or four days.

### Flow Cytometry Analysis

Analysis was performed using a CyAn flow cytometer (DakoCytomation). Cells were dissociated by short exposure to accutase or trypsin and then collected in serum free medium. ToPro-3 (Invitrogen) was added to cells at a final concentration of 0.05 nM for exclusion of dead cells.

### RT-PCR

Total RNA was prepared using RNeasy mini Kit (Qiagen) with DNasel treatment. First strand cDNA was synthesised using Superscript™ III reverse transcriptase (Invitrogen). If not otherwise specified, real time PCR was performed using Taqman® Gene Expression Assays (Applied Biosystems). Gene expression was determined relative to Gapdh using the delta Ct method. Klf4 transgene and DsRed expression were determined by standard curve calibration. All quantitative PCR reactions were performed in a 7900HT Fast Real-Time PCR system (Applied Biosystems).

### Taqman Probes:

Oct4, Mm00658129_gH; Klf4, Mm00516104_m1; Klf2, Mm01244979_g1; Klf5, Mm00456521_m1; Nanog, Mm02384862_g1; Rex1, Mm03053975_g; Fgf5, Mm00438615_m1; Lefty, Mm00438615_m1; T-Brachyury, Mm01318252_m1; Nr0b1, Mm00431729_m1; stella(Dppa3), Mm00836373_g1; Gapdh, 4352339E; β-actin, 4352341E

### Primers for Conventional RT-PCR:

Klf4-Ex1-For (5′CTTCGGACTCCGGAGGACCTTCT3′) and Klf4-Ex3-Rev (5′GCCACCGATTCCTGGTGGGTTAG3′) amplify a 390 by amplicon from endogenous Klf4. Klf4-Ex2-For (5′ATGGCTGTCAGCGACGCTCTGC3′) and Klf4-Ex3-Rev amplify a 326 by fragment from both endogenous and transgene Klf4. 137Klf4-RT-For (5′CATATCCAGTCACTATGGCTCCACC3′) and Klf4-Ex3-Rev amplify a 326 by amplicon from Klf4 transgene.
RedMST-RT-For (5′TCCGAGGACGTCATCAAGGAGTTC3′) and RedMST-RT-Rev (5′CCGATGAACTTCACCTTGTAGATGAA3′) amplify a 347 by fragment.

### Genomic PCR

Splinkerette PCR was performed as described (Mikkers et al., 2002). In brief, genomic DNA was digested with BstYI and then ligated with Splinkerette oligo adapter. The host genome and PB insertion junction was amplified with HMSP-1/PB-SP1 primers and then nested PCR using HPSP-2/PB-SP2 primers.

### Splinkerette PCR Primers

### PB-5TR-Sp1, 5′CAGTGACACTTACCGCATTGACAAGCACGC3′; PB-5TR-Sp2, 5′GAGAGAGCAATATTTCAAGAATGCATGCGT3′; PB-3TR-Sp1, 5′CCTCGATATACAGACCGATAAAACACATGC3′; PB-3TR-Sp2, 5′ACGCATGATTATCTTTAACGTACGTCACAA3′

Klf4-GT-F (5′ATGGCTGTCAGCGACGCTCTGCTCC3′) and Klf4-GT-R (5′CACCGATTCCTGGTGGGTTAGCGAGTT3′) amplify a 324 by fragment from Klf4 transgene and a 971 by fragment from Klf4 endogenous locus. PB-3TR-F (5′TTTAACCCTAGAAAGATAATCATATT3′) and PB-5TR-R (5′TTAACCCTAGAAAGATAGTCTGC3′) amplify a 583 by PB-LTR fragment created by Cre-medated deletion of Klf4 transgene.

### Immunofluorescence Staining

Cells were fixed with 4% PFA and permibilized with 0.3% Triton for antibody staining. Images were captured using a DMI4000B microscope (Leica microsystems).

Antibody details: Oct4 primary, Oct4 (C-10) (Santa Cruz, sc-5279), 1:200; secondary, Donkey anti mouse IgG Alexa 488, 1:1000; meH3K2 primary, Gift from Thomas Jenuwein, 1:1000, secondary Goat anti rabbit IgG Alexa 594 or Goat anti rabbit IgG Alexa 647

### Chimaera Production

Term chimaeras were produced by microinjection into C57BU6 blastocysts. Selected female chimaeras were mated with C57BU6J black males. Germ line transmission from cultured cell derived oocytes manifests in agouti offspring.

### Results and Discussion

We derived EpiSCs from E5.75 embryos carrying the Oct4-GFPirespac transgene (Ying et al., 2002). Cell lines were established and maintained without feeders in serum-free N2B27 medium (Ying and Smith, 2003) supplemented with activin and bFGF (Brons et al., 2007). They grow as monolayers of closely apposed cells on a fibronectin substrate (FIG. 13A). The majority of cells express the Oct4-GFP reporter (FIG. 13A). Consistent with the original descriptions (Brons et al., 2007; Tesar et al., 2007), the EpiSCs we derived express the pluripotency markers Oct4 and Nanog, but not the early epiblast marker Stella (FIG. 13B,). EpiSCs also differ from ES cells by up-regulation of post-implantation markers Fgf5, T (brachyury) and Lefty (FIG. 14).

We established both male and female EpiSC lines. Immunofluorescence revealed a prominent body of nuclear staining for the repressive histone modification trimethylated H3 lysine 27 in the female line (FIG. 13C). This is diagnostic of a silent X chromosome (Silva et al., 2003). Thus an emphatic epigenetic distinction between early and late epiblast is conserved in ES cells and EpiSCs respectively. This is reflected in a differential ability to colonise chimaeric embryos (Tesar et al., 2007). We found that after morula aggregation, Oct4GiP EpiSCs could mix with ICM cells in blastocysts, but that they quickly down-regulated GFP. Consistent with this, no contribution was detectable in egg cylinders after embryo transfer (data not shown).

EpiSCs also lose expression of Oct4 and differentiate when transferred to conventional mouse ES cell culture conditions (Brons et al., 2007). Recently, however, it has been established that small molecules that selectively inhibit the Mek/Erk MAP kinase signalling cascade and glycogen synthase kinase-3 (GSK3) provide in combination with LIF an optimal environment for derivation and propagation of ES cells from different rodent backgrounds in serum-free medium (Ying et al., 2008; Buehr, 2008; JN unpublished). The combination of two inhibitors with LIF (2i/LIF) also promotes generation of induced pluripotent stem cells (Silva et al., 2008).

We therefore tested whether EpiSCs cultured in 2i/LIF might acquire features of ground state pluripotency. However, after transfer into 2i/LIF, EpiSCs underwent massive differentiation and death such that Oct4GFP expressing cells were entirely eliminated by 3 days (FIG. 13D).

Some differentiated cells persisted, but in multiple platings of 1×10⁷EpiSCs not a single Oct4GFP expressing colony has been obtained. Since genetic background has a strong influence on the derivation and propagation of ES cells and on iPS cell generation (Batlle-Morera, 2008; Silva et al., 2008) we also examined EpiSCs from the permissive 129 strain. These EpiSCs also failed to survive in 2i/LIF.

We conclude that the EpiSC represents a stable cell state that does not naturally revert to naive pluripotent status.

The origin of ES cells and EpiSCs from early and late epiblast respectively suggests that ES cells may be capable of becoming EpiSCs. Indeed ES cells transferred into EpiSC culture conditions continued to proliferate. After passaging, cultures became relatively homogenous and EpiSC-like. Thereafter they display the marker profile of EpiSCs rather than ES cells, with maintained Oct4, reduced Nanog, and down-regulated Rex1, Nr0b1 and Klf4 (FIG. 13E, FIG. 15). Furthermore, EpiSCs derived from female ES cells show coincidence of Oct4 expression and X chromosome inactivation (FIG. 13F). This signature distinguishes EpiSCs either from ES cells or differentiated somatic cell types. To confirm that this ES cell-derived EpiSC state was truly differentiated we transferred cells back into 2i/LIF. Occasional ES cell like colonies could initially be recovered, but not after 4 or more passages in activin plus FGF (data not shown). We conclude that ES cells differentiate into EpiSCs, although a minority of undifferentiated cells persist for a while, as commonly observed in other in vitro ES cell differentiation schema (Lowell et al., 2006; Smith, 2001).

One of the genes prominently down-regulated during differentiation of ES cells into EpiSCs is Klf4. Klf4 has been implicated in ES cell self-renewal (Jiang et al., 2008; Li et al., 2005). It is induced by LIF/Stat3 signalling in ES cells, but not in EpiSCs (FIG. 16A). To test whether Klf4 might regulate the ES cell to EpiSC transition we stably transfected ES cells with a Klf4 expression plasmid. These cells show greatly reduced dependency on LIF for self-renewal, as previously reported (Li et al., 2005). On transfer to EpiSC culture conditions, however, they responded similarly to parental ES cells, growing as a monolayer and down-regulating ES cell specific marker expression while maintaining Oct4 (FIG. 16B). This indicates that forced expression of Klf4 does not prevent conversion into EpiSCs. However, even after 10 passages in activin and FGF, low frequencies of ES cell colonies were obtained upon transfer to 2i/LIF (FIG. 16C)). Therefore constitutive Klf4 either allows long-term persistence of a small fraction of undifferentiated ES cells, or enables a fraction of EpiSCs to dedifferentiate and regain the ground state.

To distinguish between these possibilities we investigated whether forced expression of Klf4 in embryo-derived EpiSCs may induce ground state pluripotency. We used PiggyBac (PB) vector-chromosome transposition to achieve high efficiency stable transfection (Wang et al., 2008). The PB vector contains independent CAG promoter units directing expression of the Klf4 open reading frame and of a DsRed reporter with linked hygromycin resistance gene (FIG. 16D). LoxP sites adjacent to the PB terminal repeats allow for excision of both expression units. The frequency of DsRed expressing cells obtained was lower after Klf4 transfection than control vector transfection (data not shown) and the level of DsRed expression was reduced. Overexpression of Klf4 may therefore be toxic to EpiSCs. Nonetheless, Klf4/DsRed expressing EpiSC cells were isolated following hygromycin selection (FIG. 16E). In activin and bFGF they did not up-regulate ES cell-specific genes (FIG. 16F) and female cells maintained an inactive X chromosome judged by me3H3K27 staining (data not shown). We conclude that expression of Klf4 at similar RNA level to that present in ES cells is not alone sufficient to reset EpiSCs and instate full pluripotency in cells maintained in activin and bFGF.

We then examined the effect of Klf4 transfection in 2i/LIF conditions, which have been shown to promote the final stages of iPS cell generation from neural stem cells and fibroblasts (Silva et al., 2008). Klf4 transfected EpiSCs were transferred into 2i/LIF 48 or 72 hours after transfection. They exhibited a wave of differentiation and cell death, similar to non-transfected EpiSCs. After 4 days in 2i/LIF, however, multiple Oct4GFP positive colonies emerged (FIG. 17A). These colonies had the tightly packed three-dimensional aggregate form typical of ES cells in 2i. They arose at a frequency between 0.1-0.2% cells surviving transfection (21 colonies from 1×10⁴ cells in one typical experiment). GFP+ve colonies were never obtained in 2i/LIF from multiple control vector transfections. Nor did undifferentiated colonies arise from EpiSCs derived from the ES cell permissive 129 strain.

We then tested whether stably transfected EpiSCs propagated in activin and FGF could convert in 2i/LIF. We found that Klf4 transfectants generated colonies in 2i/LIF with similar kinetics to cells transferred directly after transfection (FIG. 18). The yield was higher, around 1%, which could point to some element of reprogramming proceeding in the stable transfectants but could also be explained by elimination of non-transfectants and cells with toxic overexpression of Klf4. Significantly the frequency did not noticeably increase with passaging of the stable transfectants indicating that if there is any partial reprogramming this does not accumulate in the cultures.

Ten out of 12 colonies picked from a transfection with transfer to 2i/LIF after 72 hours expanded with undifferentiated morphology and stable expression of Oct4GFP. qRT-PCR analysis showed the marker profile of ES cells with up-regulation of Stella and Klf2. Conversely, Fgf5 and brachyury mRNAs were lost (FIG. 17B, FIG. 19). We examined me3H3K27 immunostaining and found that the nuclear body corresponding to the inactive X was lost in Oct4GFP positive cells after transfer to 2i/LIF (FIG. 17C). A potential disadvantage of using the PB vector and CAG promoter is that transgenes might not be silenced. However, in each of the GFP positive clones we observed partial or complete loss of visible dsRed expression (FIGS. 17D, E), although qRT-PCR analysis revealed that the transgenes were not completely silenced (FIG. 17F).

We injected cells without visible DsRed expression into C57BL/6 blastocysts. Healthy chimaeras were obtained with extensive agouti coat colour contributions (FIG. 17G). Female chimaeras mated with C57BL/6 males produced agouti offspring indicating transmission of the cultured cell genome. This confirms that developmental capacity has been fully derestricted and the authentic pluripotent state established. These cells should therefore be considered as EpiSC-derived induced pluripotent stem cells, or Epi-iPS cells.

We examined the copy number of PB integrations by genomic PCR analysis of 10 Epi-iPS cell clones. Each Epi-iPS cell line carried 1-3 PB insertions (FIG. 20A). To determine whether the low but still detectable Klf4 transgene expression may play a role in maintaining the induced phenotype we excised the transgene copies. We chose two dsRed positive clones and transfected each with a Cre expression plasmid. After 5 days, cells that no longer expressed dsRed were isolated using flow cytometry by single cell deposition into 96 well plates (FIG. 20B). Resulting clones were screened by genomic PCR for absence of the Klf4 transgene and presence of a reverted PB fragment (FIG. 20C). Two thirds of the expanded clones retained only the PB terminal repeats. RT-PCR analysis failed to detect expression of the Klf4 transgene or dsRed from these revertants (FIG. 20D). They retained ES cell morphology, Oct4GFP expression and ES cell marker profile (FIGS. 20E, F). The X chromosome silencing mark me3H3K27 was undetectable (FIG. 20G).

These cells incorporated efficiently into the ICM and subsequently the egg cylinder after morula aggregation (results not shown). We injected transgene-deleted cells into blastocysts and obtained viable high contribution chimaeras, as shown in the following Table:

TABLE
Chimaera generation from Klf4 Epi-iPS clones

| | Blastocysts | Number | Number of | Coat chimaerism | | | Germ line |
|---|---|---|---|---|---|---|---|
| Clone | injected | born | Chimaeras | High | Med | Low | transmission |
| K4C1 | 18 | 1 | 1 (died) | | 1 | | N/A |
| K4C12 | 40 | 15 | 11 | 3 | 6 | 2 | Two females |
| | | | | | | | Both 100% |
| K4C3Cre | 30 | 6 | 4 | 4 | | | One female, 50%; |
| A3 | | | | | | | 2^{nd} female lost |
| | | | | | | | litter |
| K4C3Cre | 30 | 5 | 2 (1 died) | | 1 | 1 | Not tested |
| D4 | | | | | | | |
| K4C5Cre | 46 | 7 (4 died) | 1 | | 1 | | One female |
| A5 | | | | | | | 100% |

### Table Legend.

EpiSCs are derived from hybrid 129xMF1 (Swiss albino) embryos. Chimaeras were generated in C57BL/6 blastocysts and bred with C57BU6 males. Resultant non-black pups indicate germline transmission. The cell lines are female and therefore only female chimaeras were mated.

K4C1 and K4C12 are Epi-iPS clones with Klf4 transgene; K4C3Cre and K4C5Cre clones are revertants of K4C3 and K4C5 respectively after Klf4 transgene deletion by Cre-mediated recombination.

Percentages in the germ line transmission column are of agouti pups (Epi-iPS cell derived) in the first litters.

Female chimaeras from two out of three clones produced agouti offspring in their first litter (FIG. 20H), indicative of transmission of iPS cell-derived oocytes. Therefore complete removal of the Klf4 transgene does not destabilise the induced ground state. This establishes that reprogramming has been finalised and does not depend upon ongoing transgene expression or insertional mutagenesis.

These data indicate that EpiSCs can intermingle with ICM cells during blastocyst formation, consistent with expression of the adhesion molecule Ecadherin, but differentiate or die in this environment which is reflected in loss of Oct4-GFP. Consequently EpiSCs, unlike ES cells or iPS cells, cannot participate in subsequent embryogenesis and fail to produce chimaeras (Tesar et al., 2007). The restricted potency of EpiSCs compared with ES cells appears to mirror the developmental progression from naïve pre-implantation epiblast to epithelialised egg cylinder (Rossant, 2008). ES cells in vitro recapitulate this conversion with stable alterations in gene expression, growth factor dependence, and epigenetic status. These differentiation changes may be completely reversed by re-expression of single gene that is normally down-regulated in EpiSCs. Klf4 in combination with culture in 2i/LIF revert EpiSCs to the naïve ground state. This is mediated by transcriptional resetting accompanied by activation of contrasting epigenetic processes that restrict expression of exogenous DNA sequences and erase X chromosome inactivation. Interestingly, although colonies with features of iPS cells are detectable within 7 days of Klf4 transfection, all clones recovered showed stable integration of at least one copy of the transgene, suggesting a requirement for sustained expression to effect reprogramming. However, the combination of PB-mediated low copy number integration and Cre excision allowed formal proof that once attained the Epi-iPS cell state does not require continuous presence of introduced DNA elements (Okita et al., 2008; Stadtfeld et al., 2008).

It is apparent that the exogenous transcription factor requirements for and the efficiency and kinetics of inducing pluripotency vary with the starting cell type (Aoi et al., 2008; Kim et al., 2008; Silva et al., 2008). It is striking, however, that even though other core components of the pluripotent network are already assembled, only around 1% of Klf4 expressing EpiSCs become iPS cells. This emphasizes that even though there are transcriptional similarities, EpiSCs are truly differentiated from ground state ES cells. Their reprogramming efficiency is limited in similar manner as that of somatic cells by currently unknown parameter(s).

Finally, continuous expression of Klf4 does not prevent ES cell differentiation into EpiSCs, when exposed to inductive extrinsic factors. Nonetheless, down-regulation of Klf4 may help to ensure developmental restriction of epithelialised epiblast in the embryo and safeguard against dedifferentiation to a naïve and teratogenic condition. We suggest that the creation of iPS cells may be intimately related mechanistically to the molecular transitions through which ground state pluripotency is generated and then degenerated in the early phase of mammalian embryogenesis.

In summary, mouse ES cells derived from pluripotent early epiblast contribute functionally differentiated progeny to all foetal lineages of chimaeras. In contrast, EpiSC cell lines from post-implantation epithelialised epiblast are unable to colonise the embryo even though they express core pluripotency genes, Oct4, Sox2, and Nanog. We examined interconversion between these two cell types.

ES cells can readily become EpiSCs in response to growth factor cues. In contrast, EpiSCs do not change into ES cells. We exploited PiggyBac transposition to introduce a single reprogramming factor, Klf4, into EpiSCs. No effect was apparent in EpiSC culture conditions but in ground state ES cell conditions a fraction of cells formed undifferentiated colonies. These EpiSC-derived induced pluripotent stem (Epi-iPS) cells activated expression of ES cell-specific transcripts including endogenous Klf4, and down-regulated markers of lineage specification. X chromosome silencing in female cells, a feature of the EpiSC state, was erased in Epi-iPS cells. They produced high contribution chimaeras that yielded germline transmission. These properties were maintained after Cre-mediated deletion of the Klf4 transgene, formally demonstrating complete and stable reprogramming of developmental phenotype.

These findings establish that re-expression of Klf4 in combination with an appropriate environment can regenerate the naïve ground state from EpiSCs. Reprogramming is dependent on suppression of extrinsic growth factor stimuli and proceeds to completion in less than 1% of cells, This substantiates the argument that EpiSCs are developmentally, epigenetically and functionally differentiated from ES cells. This issue acquires added significance in the light of evidence that human embryo derived stem cells are more akin to EpiSCs than to ground state ES cells (Brons et al., 2007; Rossant, 2008; Tesar et al., 2007).

### EXAMPLE 6

As shown in the Examples above, mouse EpiSCs can be converted to ground state using a single transgene (klf4; in combination with an appropriate environment) which can be deleted after the reversion.

In the present Example, the inventors show that expression from a stably integrated Nanog transgene can also convert EpiSC to ground state pluripotency.

To demonstrate this, Nanog cDNA was cloned into a piggyBac vector driven by a CAG promoter (pGG137 Nanog) (see FIG. 21). Cotransfection of pGG137 Nanog and a PB transposase expression vector to EpiSCs allows integration of pGG137 Nanog into EpiSC genome and thus enable long term expression of the Nanog transgene. We established the EpiSC cells with Nanog transgene.

After plating these cells into 2iLIF ES cell culture condition, multiple ES cells like Oct4GFP positive clones emerge after 5 days with an efficiency around 0.1-1%.

Thus stable transfection with PBNanog (and PBKlf2; results not shown) can reprogramme EpiSCs similarly to PBKlf4.

In a separate experiment, it was demonstrated that transient transfection (without PBase) using PBNanog plus PBKlf4 was also sufficient to reprogramme EpiSCs.

The results are shown in FIG. 22.

The gel image of PCR-amplified genomic DNA shows a Klf4 Epi-iPS cell clone (K4C3) sample containing the Klf4 transgene and PB LTR fragment, and two Epi-iPS cell clones (NgK4C2 and NgK4C3) generated by transient transfection with Nanog plus Klf4 that lack the Klf4 transgene and PB LTR fragment. Histograms show qRT-PCR data for two Klf2 stably transfected Epi-iPS cell clones (K2C1 and K2C2) and two Nanog+K4 transient clones. Controls are ES cells and EpiSCs.

### EXAMPLE 7

As shown in the Examples above, mouse EpiSCs can be converted to ground state using both stably or transiently expressed transgenes, in combination with 2iLIF media.

In the present Example, the inventors confirm that appropriate transgenes can also convert cells from human embryonic stem cell lines to ground state pluripotency using 2i/LIF medium.

The starting cell line for this experiment is Edi2, a human embryonic stem cell line, cultured in serum free medium with BMP4 and Fgf2. Edi2 cells were stably transfected with Nanog plus Klf4. Fluorescent imaging clearly showed expression of dsRed linked to the Nanog transgene (see FIG. 23).

The resulting cells were propagated in the presence of 2i and LIF without FGF or serum factors for over 1 month and hence are candidate ground state human cells.

### REFERENCES

Aoi T, Yae K, Nakagawa M, Ichisaka T, Okita K, et al. (2008) Generation of pluripotent stem cells from adult mouse liver and stomach cells. Science
Bain, J., Plater, L., Elliott, M., Shpiro, N., Hastie, J., McLauchlan, H., Klevernic, I., Arthur, S., Alessi, D., and Cohen, P. (2007). The selectivity of protein kinase inhibitors; a further update. Biochem J 408 297-315.
Batlle-Morera, L., Smith, A. G. and Nichols, J. (2008). Parameters influencing derivation of embryonic stem cells from murine embryos Genesis.
Beddington, R. S. P. and Robertson, E. J. (1989). An assessment of the developmental potential of embryonic stem cells in the midgestation mouse embryo. Development 105, 733-737.
Boyer, L. A., Lee, T. I., Cole, M. F., Johnstone, S. E., Levine, S. S., Zucker, J. P., Guenther, M. G., Kumar, R. M., Murray, H. L., Jenner, R. G. et al. (2005). Core transcriptional regulatory circuitry in human embryonic stem cells. Cell 122, 947-56.
Bradley, A., Evans, M. J., Kaufman, M. H. and Robertson, E. (1984). Formation of germ-line chimaeras from embryo-derived teratocarcinoma cell lines. Nature 309, 255-256.
Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., and Vallier, L. (2007). Derivation of pluripotent epiblast stem cells from mammalian embryos. Nature 448, 191-195.
Brook, F. A. and Gardner, R. L. (1997). The origin and efficient derivation of embryonic stem cells in the mouse. PNAS 94, 5709-5712.
Burdon, T., Stracey, C., Chambers, I., Nichols, J. and Smith, A. (1999). Suppression of SHP-2 and ERK signalling promotes self-renewal of mouse embryonic stem cells. Dev Biol 210, 30-43.
Buehr M, Meek S, Blair K, Yang J, Ure J, Silva J, McLay R, Hall J, Ying Q, Smith A. Capture of Authentic Embryonic Stem Cells from Rat Blastocysts. Cell. 2008 Dec. 26; 135(7):1287-98.
Conti, L., Pollard, S. M., Gorba, T., Reitano, E., Toselli, M., Biella, G., Sun, Y., Sanzone, S., Ying, Q. L., Cattaneo, E., and Smith, A. (2005). Niche-independent symmetrical self-renewal of a mammalian tissue stem cell. PLoS Biol 3, e283.
Evans, M. J. and Kaufman, M. H. (1981). Establishment in culture of pluripotential cells from mouse embryos. Nature 292, 154-6.
Gardner, R. L. and Rossant, J. (1979). Investigation of the fate of 4-5 day post-coitum mouse inner cell mass cells by blastocyst injection. J Embryol Exp Morphol 52, 141-52.
Jiang, J., Chan, Y. S., Loh, Y. H., Cai, J., Tong, G. Q., Lim, C. A., Robson, P., Zhong, S. and Ng, H. H. (2008). A core Klf circuitry regulates self-renewal of embryonic stem cells. Nat Cell Biol 10, 353-60.
Kim J B, Zaehres H, Wu G, Gentile L, Ko K, et al. (2008) Pluripotent stem cells induced from adult neural stem cells by reprogramming with two factors. Nature 454: 646-650.
Kunath, T., Saba-El-Leil, M. K., Almousailleakh, M., Wray, J., Meloche, S. and Smith, A. (2007). FGF stimulation of the Erk1/2 signalling cascade triggers transition of pluripotent embryonic stem cells from self-renewal to lineage commitment. Development 134, 2895-902.
Li, Y., McClintick, J., Zhong, L., Edenberg, H. J., Yoder, M. C. and Chan, R. J. (2005). Murine embryonic stem cell differentiation is promoted by SOCS-3 and inhibited by the zinc finger transcription factor Klf4. Blood 105, 635-7.
Loh, Y. H., Wu, Q., Chew, J. L., Vega, V. B., Zhang, W., Chen, X., Bourque, G., George, J.,
Leong, B., Liu, J. et al. (2006). The Oct4 and Nanog transcription network regulates pluripotency in mouse embryonic stem cells. Nat Genet 38, 431-40.
Lowell, S., Benchoua, A., Heavey, B. and Smith, A. G. (2006). Notch promotes neural lineage entry by pluripotent embryonic stem cells. PLoS Biol 4, 805-818.
Maherali, N., Sridharan, R., Xie, W., Utikal, J., Eminli, S., Arnold, K., Stadtfeld, M., Yachechko, R., Tchieu, J., Jaenisch, R., et al. (2007). Directly Reprogrammed Fibroblasts Show Global Epigenetic Remodeling and Widespread Tissue Contribution. Cell Stem Cell 1, 55-70.
Martin, G. R. (1981). Isolation of a pluripotent cell line from early mouse embryos cultured in medium conditioned by teratocarcinoma stem cells. Proc. Natl. Acad. Sci. USA 78, 7634-7638.
Meissner, A., Wernig, M., and Jaenisch, R. (2007). Direct reprogramming of genetically unmodified fibroblasts into pluripotent stem cells. Nat Biotechnol.
Morita, S., Kojima, T., and Kitamura, T. (2000). Plat-E: an efficient and stable system for transient packaging of retroviruses. Gene Ther 7, 1063-1066.
Nagy, A., Rossant, J., Nagy, R., Abramow-Newerly, W. and Roder, J. C. (1993). Derivation of completely cell culture-derived mice from early-passage embryonic stem cells. Proc Natl Acad Sci USA 90, 8424-8.
Nakagawa M, Koyanagi M, Tanabe K, Takahashi K, Ichisaka T, et al. (2008) Generation of induced pluripotent stem cells without Myc from mouse and human fibroblasts. Nat Biotechnol 26: 101-106.
Okita, K., Ichisaka, T., and Yamanaka, S. (2007). Generation of germline-competent induced pluripotent stem cells. Nature 448, 313-317.
Okita, K., Nakagawa, M., Hyenjong, H., Ichisaka, T. and Yamanaka, S. (2008). Generation of mouse induced pluripotent stem cells without viral vectors. Science 322, 949-53.
Rossant, J. (2008). Stem cells and early lineage development. Cell 132, 527-31.
Pollard, S. M., Conti, L., Sun, Y., Goffredo, D. & Smith, A. Adherent neural stem (NS) cells from fetal and adult forebrain. Cereb Cortex 16 Suppl 1, i112-20 (2006).
Rossant, J., Gardner, R. L. and Alexandre, H. L. (1978). Investigation of the potency of cells from the postimplantation mouse embryo by blastocyst injection: a preliminary report. J Embryol Exp Morphol 48, 239-47.
Silva, J., Chambers, I., Pollard, S., and Smith, A. (2006). Nanog promotes transfer of pluripotency after cell fusion. Nature 441, 997-1001.
Silva, J., Mak, W., Zvetkova, I., Appanah, R., Nesterova, T. B., Webster, Z., Peters, A. H., Jenuwein, T., Otte, A. P., and Brockdorff, N. (2003). Establishment of histone h3 methylation on the inactive X chromosome requires transient recruitment of Eed-Enx1 polycomb group complexes. Dev Cell 4, 481-495.
Silva, J., Barrandon, O., Nichols, J., Kawaguchi, J., Theunissen, T. W. and Smith, A. (2008). Promotion of reprogramming to ground state pluripotency by signal inhibition. PLoS Biol 6, e253.
Smith, A. G. (2001). Embryo-derived stem cells: of mice and men. Ann. Rev. Cell Dev. Biol. 17, 435-462.
Smith, A. G., Heath, J. K., Donaldson, D. D., Wong, G. G., Moreau, J., Stahl, M. and Rogers, D. (1988). Inhibition of pluripotential embryonic stem cell differentiation by purified polypeptides. Nature 336, 688-690.
Stadtfeld, M., Nagaya, M., Utikal, J., Weir, G. and Hochedlinger, K. (2008). Induced pluripotent stem cells generated without viral integration. Science 322, 945-9.
Stpyridis, M. P., Lunn, J. S., Collins, B. J. and Storey, K. G. (2007). A discrete period of FGF-induced Erk1/2 signalling is required for vertebrate neural specification. Development 134, 2889-94.
Sun, Y., Pollard, S., Conti, L., Toselli, M., Biella, G., Parkin, G., Willatt, L., Falk, A., Cattaneo, E., and Smith, A. (2008). Long-term tripotent differentiation capacity of human neural stem (NS) cells in adherent culture. Mol Cell Neurosci in press.
Takahashi, K., and Yamanaka, S. (2006). Induction of pluripotent stem cells from mouse embryonic and adult fibroblast cultures by defined factors. Cell 126, 663-676.
Tesar, P. J., Chenoweth, J. G., Brook, F. A., Davies, T. J., Evans, E. P., Mack, D. L., Gardner, R. L., and McKay, R. D. (2007). New cell lines from mouse epiblast share defining features with human embryonic stem cells. Nature 448, 196-199.
Wang, J., Rao, S., Chu, J., Shen, X., Levasseur, D. N., Theunissen, T. W. and Orkin, S. H. (2006). A protein interaction network for pluripotency of embryonic stem cells. Nature 444, 364-8.
Wang, W., Lin, C., Lu, D., Ning, Z., Cox, T., Melvin, D., Wang, X., Bradley, A. and Liu, P. (2008). Chromosomal transposition of PiggyBac in mouse embryonic stem cells. Proc Natl Acad Sci USA 105, 9290-5.
Wernig, M., Meissner, A., Foreman, R., Brambrink, T., Ku, M., Hochedlinger, K., Bernstein, B. E., and Jaenisch, R. (2007). In vitro reprogramming of fibroblasts into a pluripotent ES-cell-like state. Nature 448, 318-324.
Williams, R. L., Hilton, D. J., Pease, S., Willson, T. A., Stewart, C. L., Gearing, D. P., Wagner, E. F., Metcalf, D., Nicola, N. A. and Gough, N. M. (1988). Myeloid leukaemia inhibitory factor maintains the developmental potential of embryonic stem cells. Nature 336, 684-687.
Ying, Q. L., Wray, J., Nichols, J., Batlle-Morera, L., Doble, B., Woodgett, J., Cohen, P. and Smith, A. (2008). The ground state of embryonic stem cell self-renewal. Nature 453, 519-23.
Ying, Q. L., Nichols, J., Evans, E. P., and Smith, A. G. (2002). Changing potency by spontaneous fusion. Nature 416, 545-548.
Ying, Q. L., and Smith, A. G. (2003). Defined conditions for neural commitment and differentiation. Methods Enzymol 365, 327-341.
Ying, Q. L., Nichols, J., Chambers, I. and Smith, A. (2003). BMP induction of 1d proteins suppresses differentiation and sustains embryonic stem cell self-renewal in collaboration with STAT3. Cell 115, 281-92.
Yu, J. et al. Induced pluripotent stem cell lines derived from human somatic cells. Science 318, 1917-20 (2007).

1. A method of reprogramming, comprising:—
(a) providing a cell to be reprogrammed;
(b) introducing into the cell a nucleic acid or protein preparation which expresses or provides one or more reprogramming factors, wherein the cell is reprogrammed into a partially reprogrammed cell by the reprogramming factors;
(C) culturing the partially reprogrammed cell in the presence of an inhibitor of the MAPK cascade and an activator or Stat3, optionally with a GSK3 inhibitor, wherein the partially reprogrammed cell is induced to a state of pluripotency; and
(c) thereby obtaining a fully reprogrammed cell

2. A method of reprogramming comprising:—
(a) providing a cell to be reprogrammed;
(b) transiently expressing one or more reprogramming factors in the cells whereby the cell is reprogrammed; and
(c) maintaining the reprogrammed cell in culture until extrachromosomal genetic material, if any, introduced during step (b) is lost, thereby providing a reprogrammed cell which is not genetically modified compared to the cell of step (a).

3. A method of reprogramming, comprising:—
(a) providing a cell to be reprogrammed;
(b) introducing extrachromosomal genetic material into the cell so that one or more reprogramming factors are transiently expressed in the cell whereby the cell is reprogrammed; and
(c) maintaining the reprogrammed cell in culture until the extrachromosomal genetic material is lost, whereby a reprogrammed cell is obtained being not genetically modified compared to the cell of step (a).

4. A method according to claim 1 wherein the nucleic acid preparation is integrated into the genome, and wherein the method optionally comprises the step of excising the nucleic acid encoding the reprogramming factor.

5. A method according to any of claims 1-3, comprising introducing into the cells a plasmid preparation which expresses one or more reprogramming factors in the cell.

6. A method according to any of claims 1-4, wherein the nucleic acid preparation comprises one or more plasmids which express in the cell one or more reprogramming factors selected from Oct3/4, Sox2, Klf4, c-Myc, Nanog and LIN28.

7. A method according to claim 6 wherein the nucleic acid preparation does not include a plasmid which expresses in the cell Sox2.

8. A method according to claim 7 wherein the nucleic acid preparation expresses only the reprogramming factors Oct4 and Klf4 or Nanog and Klf4.

9. A method according to any one of claims 1-5 wherein the nucleic acid preparation expresses only the reprogramming factor Klf4 or Klf2, or only Nanog.

10. A method according to any of claims 1-9 wherein the cell to be reprogrammed is a somatic tissue stem cell.

11. A method according to claim 10 wherein the cell to be reprogrammed is an adult somatic tissue stem cell.

12. A method according to claim 10 or claim 11 wherein the cell to be reprogrammed is a neural stem cell.

13. A method according to any of claims 1-9 wherein the cell to be reprogrammed is an Epistem cell (EpiSc).

14. A method according to any of claims 1-13, wherein the cell to be reprogrammed is a mouse cell.

15. A method according to any of claims 1-14 wherein the cell to be reprogrammed is a human cell.

16. A method according to any of claims 1-15, comprising culturing the cell in medium comprising one or more kinase inhibitors which inhibits a kinase responsible for an intracellular signalling cascade.

17. A method according to claim 16 wherein the intracellular signalling cascade is the ERK1 or ERK2 cascade.

18. A method according to any of claims 1-17, comprising culturing the cell in medium comprising a MEK inhibitor.

19. A method according to any of claims 1-18 comprising culturing the cell in medium comprising a MEK inhibitor and an activator of Stat3 or gp130 signalling, optionally with a GSK3 inhibitor.

20. A method according to claim 19 wherein the activator of Stat3 or gp130 signalling is LIF.

21. A method according to any one of claims 1-20, comprising:—
(a) providing a cell to be reprogrammed;
(b) introducing into the cell a plasmid preparation which expresses one or more reprogramming factors;
(c) culturing the cell in the presence of one or more factors which promote growth of the cell to be reprogrammed;
(d) culturing the cell from (c) in the presence of one or more factors which promote the growth of a reprogrammed cell;
(e) culturing the cell from (d) in the presence of a MEK inhibitor and optionally a GSK3 inhibitor; and
(f) thereby obtaining a reprogrammed cell

22. A method according to claim 21, comprising in step (d) culturing the cell from (c) in the presence of LIF.

23. A method according to claim 21 or 22 for reprogramming of a neural cell, comprising in step (c) culturing the neural cell in the presence of EGF and FGF.

24. A method according to any one of claims 1-23 wherein the cell to be reprogrammed comprises a silenced X chromosome and the reprogramming causes reactivation of the silenced X chromosome.

25. A method according to any one of claims 1-24 wherein the non-human cell to be reprogrammed is incapable of yielding chimaeras on blastocyst injection and the reprogramming imparts the capability to yield chimaeras on blastocyst injection.

26. An isolated mouse pluripotent stem cell obtained by a method according to any of claim 1-14 or 16-25 as dependent on claim 14.

27. An isolated human pluripotent stem cell obtained by a method according to any of claim 1-13, 15, or 16-25 as dependent on claim 15.

28. An isolated human pluripotent stem cell, characterised in that:—
(i) it can be maintained in a pluripotent state in culture medium containing a MEK inhibitor and a GSK3 inhibitor; and
(ii) it does not require FGF signalling in order to self-renew.

29. A cell according to any one of claims 26-28, further characterised in that it has two active X chromosomes.

30. A cell according to any one of claims 26-29, which differentiates in culture medium containing FGF.

31. A cell according to any of claims 26-30, characterized by expression of one or more markers specific to pre-implantation embryonic stem cells.

32. A cell according to any of claims 26-31, characterized by absence of expression of markers of epistem cell phenotype.

33. A cell according to any of claims 26-32 which self-renews in culture medium free of serum and containing LIF and BMP.

34. A population of cells comprising at least 10, 20, 30, 40, 50, 60, 70, 80, 90%, or consisting of 100%, cells according to any of claims 26-33.

35. A method of converting a partially reprogrammed cell into a fully reprogrammed, pluripotent cell, comprising maintaining the partially reprogrammed cell in culture medium containing one or more kinase inhibitors which inhibits a kinase responsible for an intracellular signalling cascade, which is optionally the ERK1 or ERK2 cascade.

36. A method according to claim 35 wherein the inhibitors comprise or consist of: (i) a MEK inhibitor and optionally a GSK 3 inhibitor.

37. A method according to claim 35 wherein the medium further comprises an inhibitor of gp130 signalling which is optionally LIF.

38. A method according to any one of claims 35-37 wherein the partially reprogrammed cell is obtained by expression in a somatic cell of one or more reprogramming factors.

39. A method according to claim 38 wherein the expression is retroviral expression or plasmid expression.

40. A method according to any of claims 35-39 wherein the partially reprogrammed cell is derived from a somatic tissue stem cell.

41. A method according to claim 40 wherein the partially reprogrammed cell is derived from an adult somatic tissue stem cell.

42. A method according to any one of claims 35-41 wherein the partially reprogrammed cell is derived from an epistem cell.

43. A method of converting a human epistem cell expressing or which has expressed one or more reprogramming factors into a pluripotent cell having the characteristics of a pre-implantation pluripotent stem cell, comprising maintaining the human epistem cell in culture medium containing (i) a MEK inhibitor, (ii) a GSK 3 inhibitor, or (iii) both a MEK inhibitor and a GSK 3 inhibitor and optionally LIF; wherein the one or more reprogramming factors optionally comprises Klf4 or Klf2 encoded by heterologous nucleic acid.

44. A method according to claim 42 or claim 43 wherein the human epistem cell is obtained from a human embryo.

45. A method according to claim 42 or claim 43 wherein the human epistem cell is obtained from a human somatic cell.

46. A method of activating an X chromosome in a pluripotent cell having two X chromosomes, one of which is inactive, comprising culturing the pluripotent cell in medium containing one or more kinase inhibitors which inhibits a kinase responsible for an intracellular signalling cascade.

47. A method according to claim 46 wherein the intracellular signalling cascade is the ERK1 or ERK2 cascade.

48. A method according to claim 46 or claim 47 wherein the kinase inhibitor is (i) a MEK inhibitor, or (ii) both a MEK inhibitor and a GSK 3 inhibitor, whereby a pluripotent cell is obtained having two active X chromosomes.

49. A method according to claim 48 wherein the medium comprises an activator of Stat3 or gp130 signalling.

50. A method according to claim 49 wherein the activator of Stat3 or gp130 signalling is LIF.

51. A method according to any one of claims 46-50, wherein the cell is a human cell.

52. A method of reprogramming a human cell substantially as hereinbefore described with reference to any of the Examples herein.

53. A reprogrammed human cell substantially as hereinbefore described with reference to any of the Examples herein.

54. A method of reprogramming a cell, comprising:—
(a) providing a cell to be reprogrammed;
(b) introducing into the cell heterologous nucleic acid encoding one or more reprogramming factors, wherein the cell is reprogrammed into a reprogrammed cell by expression of the reprogramming factors; and
(c) thereby obtaining a reprogrammed cell
wherein the reprogramming factors are selected from Oct3/4, Klf4, c-Myc, Nanog and LIN28,
with the proviso that the nucleic acid does not encode Sox2.

55. A method according to claim 54 wherein the cell to be reprogrammed is a somatic tissue stem cell.

56. A method according to claim 55 wherein the cell to be reprogrammed is an adult somatic tissue stem cell.

57. A method according to claim 55 or claim 56 wherein the cell to be reprogrammed is a neural stem cell or epistem cell.

58. A method according to any one of claims 54 to 57 wherein only two reprogramming factors are used, one of which is Oct3/4.

59. A method according to claim 58 wherein the only second factor is Klf4 or c-Myc.

60. A method according to claim 58 or claim 59 wherein the nucleic acid encodes only the reprogramming factors Oct4 and Klf4 or Nanog and Klf4.

61. A method according to any one of claims 54 to 57 wherein the nucleic acid encodes only the reprogramming factor Klf4 or Klf2.

62. Use of a medium comprising:
(i) an inhibitor of the ERK1 or ERK2 intracellular signalling cascade which is optionally a MEK inhibitor and optionally a GSK3 inhibitor, and
(ii) an activator of Stat3, which is optionally LIF,
in a method as claimed in any one of claims 1 to 25 or 35 to 45 or 54 to 57, for induction of complete reprogramming to pluripotency of the cell to be reprogrammed.

63. Use of a medium comprising:
(i) an inhibitor of the ERK1 or ERK2 intracellular signalling cascade which is optionally a MEK inhibitor, and optionally a GSK3 inhibitor, and
(ii) an activator of Stat3, which is optionally LIF,
in the preparation of an agent for induction of complete reprogramming to pluripotency of a cell to be reprogrammed.

64. Use of a medium comprising:
(i) an inhibitor of the ERK1 or ERK2 intracellular signalling cascade which is optionally a MEK inhibitor, and optionally a GSK3 inhibitor, and
(ii) an activator of Stat3, which is optionally LIF,
in the preparation of an agent for induction of conversion of a cell having the characteristics of an epistem cell into a pluripotent cell.

65. Use of a medium comprising:
(i) a MEK inhibitor, a GSK3 inhibitor or both a MEK inhibitor and a GSK3 inhibitor, and
(ii) LIF,
in a method as claimed in any one of claims 43 to 45 for induction of conversion of the human epistem cell into a pluripotent cell.

66. Use as claimed in any of claims 62 to 65 wherein the cells are cultured in the medium.

67. Use as claimed in any of claims 62 to 66 wherein the medium is applied directly to the cells, optionally without culture on feeder.

68. A method of reprogramming an epistem cell (EpiSC), the method comprising:—
(a) providing an EpiSC cell to be reprogrammed;
(b) introducing into the cell a nucleic acid or protein preparation which expresses or provides one or more reprogramming factors.

69. A method according to claim 68 wherein the EpiSC is reprogrammed to ground state pluripotency.

70. A method according to claim 68 or 69 wherein nucleic acid which expresses the one or more reprogramming factors is introduced

71. A method according to claim 70 wherein the nucleic acid preparation is integrated into the genome, and wherein the method optionally comprises the step of excising the nucleic acid encoding the reprogramming factor.

72. A method according to claim 70 wherein the nucleic acid preparation is expressed transiently.

73. A method according to any of claims 68-72, wherein only two reprogramming factors are introduced or expressed.

74. A method according to claim 73 wherein the two reprogramming factors are introduced Nanog and Klf4, or Nanog and Klf2.

75. A method according to any of claims 68-72, wherein only one reprogramming factor is introduced or expressed, wherein the one reprogramming factor is selected from: Nanog, Klf4, or Klf2.

76. A method according to any of claims 68-75, wherein the EpiSc is cultured in medium comprising a Stat3 activating cytokine.

77. A method according to claim 76 wherein the Stat3 activating cytokine is LIF.
